SEQUENCE LISTING (19 sequences)

SEQ ID NO: 1 - DNA, 1486 bp, Homo sapien
  CDS (47)...(1084)
gaattcggcacgagggcaggaggtagacacggcacagggcgccgagatgcggcggggcgcgggcgcggctcggggacgcgcttcctggtgctgggcgctggcgctgctttggctcgcggtggttccgggctggtcccgggtctcgggcatcccctcccggcgccactggccggtgccctacaagcgctttgacttccgtccaaaacctgatccttattgtcaagctaagtatactttctgtccaactggctcacctatcccagttatggagggtgatgatgacattgaagtttttcgattacaagccccagtatgggaatttaaatatggagacctcctgggacacttgaaaattatgcatgatgccattggattcagaagtacattaactggcaagaactacacaatggaatggtatgaacttttccaacttggcaactgtacatttccccatctccgacctgaaatggatgcccctttctggtgtaatcaaggcgctgcctgcttttttgagggaattgatgatgttcactggaaggaaaatgggacattagttcaagtagcaactatatcaggaaacatgttcaaccaaatggcaaagtgggtgaaacaggacaatgaaacaggaatttattatgagacatggaatgtaaaagccagcccagaaaagggggcagagacatggtttgattcctacgactgttccaaatttgtgttaaggacctttaacaagttggctgaatttggagcagagttcaagaacatagaaaccaactatacaagaatatttctttacagtggagaacctacttatctgggaaatgaaacatctgtttttgggccaacaggaaacaagactcttggtttagccataaaaagattttattaccccttcaaaccacatttgccaactaaagaatttctgttgagtctcttgcaaatttttgatgcagtgattgtgcacaaacagttctatttgttttataattttgaatattggtttttacctatgaaattcccttttattaaaataacatatgaagaaatccctttacctatcagaaacaaaacactctctggtttataaaacaccttaattctactgctctttttttctccaatcaccagcatctgtttttcagggggtgattttacttttgtgaattccttagcctttcttccttggtgcataaagttaaaatgcacatcagcagaattgctgcatattaacatctcaggactcttctcttgtaaagaagctgaaattcgtactatattggccaaagtgagcgagttaggtgatcttggtttcaatttccgagcctttgttaatatggagaattatggttcatatcagttatgtaggacctttggacccagggtcctacagatagatatggtgtgcccagattttaaaaataccttcaaaaataaaaaatacattcagtgacaaaaaaaaaaaaaaaaaaatagcggccgcctcgag

SEQ ID NO: 2 - protein, 346 aa, Homo sapien
MRRGAGAARGRASWCWALALLWLAVVPGWSRVSGIPSRRHWPVPYKRFDFRPKPDPYCQAKYTFCPTGSPIPVMEGDDDIEVFRLQAPVWEFKYGDLLGHLKIMHDAIGFRSTLTGKNYTMEWYELFQLGNCTFPHLRPEMDAPFWCNQGAACFFEGIDDVHWKENGTLVQVATISGNMFNQMAKWVKQDNETGIYYETWNVKASPEKGAETWFDSYDCSKFVLRTFNKLAEFGAEFKNIETNYTRIFLYSGEPTYLGNETSVFGPTGNKTLGLAIKRFYYPFKPHLPTKEFLLSLLQIFDAVIVHKQFYLFYNFEYWFLPMKFPFIKITYEEIPLPIRNKTLSGL

SEQ ID NO: 3 - DNA, 18 bp, Artificial Sequence
  Oligonucleotide ZC976
cgttgtaaaacgacggcc

SEQ ID NO: 4 - DNA, 17 bp, Artificial Sequence
  Oligonucleotide ZC447
taacaatttcacacagg

SEQ ID NO: 5 - DNA, 20 bp, Artificial Sequence
  Oligonucleotide ZC14487
gacttccgtccaaaacctga

SEQ ID NO: 6 - DNA, 20 bp, Artificial Sequence
  Oligonucleotide ZC14716
aggggcatccatttcaggtg

SEQ ID NO: 7 - DNA, 20 bp, Artificial Sequence
  Oligonucleotide ZC14712
atggctaaaccaagagtctt

SEQ ID NO: 8 - DNA, 20 bp, Artificial Sequence
  Oligonucleotide ZC14710
gggtgaaacaggacaatgaa

SEQ ID NO: 9 - DNA, 20 bp, Artificial Sequence
  Oligonucleotide ZC14488
ttatgcaccaaggaagaaag

SEQ ID NO: 10 - DNA, 20 bp, Artificial Sequence
  Oligonucleotide ZC14711
ttttctccaatcaccagcat

SEQ ID NO: 11 - DNA, 18 bp, Artificial Sequence
  Oligonucleotide ZC14430
gtacatttccccatctcc

SEQ ID NO: 12 - DNA, 18 bp, Artificial Sequence
  Oligonucleotide ZC14431
ccattttccttccagtga

SEQ ID NO: 13 - DNA, 1038 bp, Artificial Sequence
  Degenerate nucleotide sequence encoding zsig46 polypeptide of SEQ ID NO2
atgmgnmgnggngcnggngcngcnmgnggnmgngcnwsntggtgytgggcnytngcnytnytntggytngcngtngtnccnggntggwsnmgngtnwsnggnathccnwsnmgnmgncaytggccngtnccntayaarmgnttygayttymgnccnaarccngayccntaytgycargcnaartayacnttytgyccnacnggnwsnccnathccngtnatggarggngaygaygayathgargtnttymgnytncargcnccngtntgggarttyaartayggngayytnytnggncayytnaarathatgcaygaygcnathggnttymgnwsnacnytnacnggnaaraaytayacnatggartggtaygarytnttycarytnggnaaytgyacnttyccncayytnmgnccngaratggaygcnccnttytggtgyaaycarggngcngcntgyttyttygarggnathgaygaygtncaytggaargaraayggnacnytngtncargtngcnacnathwsnggnaayatgttyaaycaratggcnaartgggtnaarcargayaaygaracnggnathtaytaygaracntggaaygtnaargcnwsnccngaraarggngcngaracntggttygaywsntaygaytgywsnaarttygtnytnmgnacnttyaayaarytngcngarttyggngcngarttyaaraayathgaracnaaytayacnmgnathttyytntaywsnggngarccnacntayytnggnaaygaracnwsngtnttyggnccnacnggnaayaaracnytnggnytngcnathaarmgnttytaytayccnttyaarccncayytnccnacnaargarttyytnytnwsnytnytncarathttygaygcngtnathgtncayaarcarttytayytnttytayaayttygartaytggttyytnccnatgaarttyccnttyathaarathacntaygargarathccnytnccnathmgnaayaaracnytnwsnggnytn

SEQ ID NO: 14 - protein, 7 aa, Artificial Sequence
  Glu-Glu affinity tag peptide
EEYMPME

SEQ ID NO: 15 - DNA, 25 bp, Artificial Sequence
  Oligonucleotide ZC14695
taactcgaggagatgcggcggggcg

SEQ ID NO: 16 - DNA, 30 bp, Artificial Sequence
  Oligonucleotide ZC15231
ttcggatcctaaaccagagagtgttttgtt

SEQ ID NO: 17 - DNA, 24 bp, Artificial Sequence
  Oligonucleotide ZC14696
ttaggatcctggtcccgggtctcg

SEQ ID NO: 18 - DNA, 30 bp, Artificial Sequence
  Oligonucleotide ZC15230
ttcctcgaggttttataaaccagagagtgt

SEQ ID NO: 19 - protein, 6 aa, Artificial Sequence
  Glu-Glu tag peptide
EYMPVD

(19) United States
(12) Patent Application Publication
(10) Pub. No.: US 2002/0173624 A1
(43) Pub. Date: Nov. 21, 2002

(54) Secreted proteins encoded by human chromosome 13

(75) Inventors: Paul O. Sheppard, Granite Falls, WA (US); Debra G. Gilbertson, Seattle, WA (US)

Correspondence Address: Brian J. Walsh, ZymoGenetics, Inc., 1201 Eastlake Avenue East, Seattle, WA, 98102, US

(73) Assignee: ZymoGenetics, Inc.

(21) Appl. No.: 10/010,050

(22) Filed: Nov. 9, 2001

Related U.S. Application Data
(63) Continuation of application No. 09/122,383, filed on Jul. 24, 1998.
(60) Provisional application No. 60/053,613, filed on Jul. 24, 1997.

(51) Int. Cl. C07K 14/435; C12P 21/02; C12N 5/06; C07H 21/04

(52) U.S. Cl. 530/350; 435/69.1; 435/325; 435/320.1; 536/23.5

(57) ABSTRACT

The present invention relates to polynucleotide and polypeptide molecules a for zsig46 polypeptide, a novel secreted protein located on human chromosome 13. The zsig46 polypeptides, and polynucleotides encoding them, are secreted proteins and may be used in the study of receptors for which a ligand has not yet been identified, of secretory pathways and the like. The present invention also includes antibodies to the zsig46 polypeptides.

### REFERENCE TO RELATED APPLICATIONS

This application is a continuation of U.S. application Ser. No. 09/122,383 (filed Jul. 24, 1998), which claims the benefit of U.S. Provisional Application 60/053,613 (filed Jul. 24, 1997), the contents of which are incorporated by reference.

### BACKGROUND OF THE INVENTION

The goal of the human genome project is to sequence the entire human genome. Databases, both public and private, are routinely updated with the latest sequencing data. Hawkins et al., submitted a genomic sequence of human chromosome 13, bases 1 to 106,988, to such a database. Identification of genes located on human chromosome 13, particularly those encoding secreted proteins, will further the understanding of normal and abnormal human physiology.

The present invention provides novel secreted polypeptides encoded by human chromosome 13 for these and other uses that should be apparent to those skilled in the art from the teachings herein.

### SUMMARY OF THE INVENTION

Within one aspect the invention provides an isolated polypeptide comprising a sequence of amino acid residues that is at least 80% identical in amino acid sequence to residues 31-346 of SEQ ID NO:2, wherein the sequence comprises cysteine residues corresponding to residues 58, 65, 132, 147, 153 and 219 of SEQ ID NO:2. Within one embodiment, the polypeptide is at least 90% identical in amino acid sequence to residues 29-346 of SEQ ID NO:2, wherein the sequence comprises cysteine residues corresponding to residues 58, 65, 132, 147, 153 and 219 of SEQ ID NO:2. Within another embodiment the polypeptide comprises residues 1-346 of SEQ ID NO:2. Within another embodiment the polypeptide is covalently linked amino terminally or carboxy terminally to a moiety selected from the group consisting of: affinity tags, toxins, radionucleotides, enzymes and fluorophores.

Within another aspect is provided an isolated polypeptide having a sequence of amino acid residues selected from the group consisting of: (a) a sequence of amino acid residues from amino acid residue 1, 29 or 31 to amino acid residue 37 of SEQ ID NO: 2; (b) a sequence of amino acid residues from amino acid residue 40 or 48 to amino acid residue 346 of SEQ ID NO: 2; (c) a sequence of amino acid residues from amino acid residue 29, 31 or 40 to amino acid residue 45 of SEQ ID NO: 2; (d) a sequence of amino acid residues from amino acid residues 29, 31, 40 or 48 to amino acid residue 276 of SEQ ID NO:2; (e) a sequence of amino acid residues from amino acid residue 278 to amino acid residue 346 of SEQ ID NO: 2; and f) a sequence of amino acid residues that is at least 80% identical in amino acid sequence to a), b), c), d) or e).

Within another aspect is provided a fusion protein consisting essentially of a first portion and a second portion joined by a peptide bond, the first portion comprising a polypeptide that is at least 80% identical in amino acid sequence to the amino acid sequence of a polypeptide selected from the group consisting of: a) a sequence of amino acid residues from amino acid residue 1, 29 or 31 to amino acid residue 37 of SEQ ID NO: 2; b) a sequence of amino acid residues from amino acid residue 40 or 48 to amino acid residue 346 of SEQ ID NO: 2; c) a sequence of amino acid residues from amino acid residue 29, 31 or 40 to amino acid residue 45 of SEQ ID NO: 2; d) a sequence of amino acid residues from amino acid residues 29, 31, 40 or 48 to amino acid residue 276 of SEQ ID NO:2; e) a sequence of amino acid residues from amino acid residue 278 to amino acid residue 346 of SEQ ID NO: 2; f) a sequence of amino acid residues from amino acid residue 1, 29 or 31 to amino acid residue 356 of SEQ ID NO:2; and g) a sequence of amino acid residues that is at least 80% identical in amino acid sequence to a), b), c), d) e) or f); and the second portion comprising another polypeptide.

Within another aspect is provided a fusion protein comprising a secretory signal sequence having the amino acid sequence of amino acid residues 1-28 or 1-30 of SEQ ID NO:2, wherein the secretory signal sequence is operably linked to an additional polypeptide.

Within yet another aspect is provided an isolated protein comprising a first polypeptide that is at least 80% identical in amino acid sequence to the amino acid sequence of a polypeptide selected from the group consisting of: a) amino acid residues 1-29 or 30 of SEQ ID NO:2, the polypeptide comprising a cysteine residue at a position corresponding to residue 15 of SEQ ID NO:2; b) amino acid residues 48-276 of SEQ ID NO:2, the polypeptide comprising cysteine residues at positions corresponding to residues 58, 65, 132, 147, 153 and 219 of SEQ ID NO:2; and c) amino acid residues 31-346 of SEQ ID NO:2, the polypeptide comprising cysteine residues at positions corresponding to residues 58, 65, 132, 147, 153 and 219 of SEQ ID NO:2; complexed to a second polypeptide. Within one embodiment the polypeptides are complexed by intermolecular disulfide bonds. Within another embodiment the protein is a homodimer. Within yet another embodiment the protein is a heterodimer.

Within another aspect is provided an expression vector comprising the following operably linked elements: a transcription promoter; a DNA segment encoding a polypeptide comprising a sequence of amino acid residues that is at least 80% identical in amino acid sequence to residues 31-346 of SEQ ID NO:2, wherein the sequence comprises cysteine residues corresponding to residues 58, 65, 132, 147, 153 and 219 of SEQ ID NO:2; and a transcription terminator. Within one embodiment the DNA segment encodes a polypeptide that is at least 90% identical in amino acid sequence to residues 29-346 of SEQ ID NO:2, wherein the sequence comprises cysteine residues corresponding to residues 58, 65, 132, 147, 153 and 219 of SEQ ID NO:2. Within another embodiment the DNA segment encodes a polypeptide comprising residues 1-346 of SEQ ID NO:2. Within another embodiment the DNA segment encodes a polypeptide covalently linked amino terminally or carboxy terminally to an affinity tag. Within yet another embodiment the DNA segment further encodes a secretory signal sequence operably linked to the polypeptide. Within a related embodiment the secretory signal sequence comprises residues 1-28 or 1-30 of SEQ ID NO:2.

In yet another aspect is provided a cultured cell into which has been introduced an expression vector comprising the following operably linked elements: a transcription promoter; a DNA segment encoding a polypeptide comprising a sequence of amino acid residues that is at least 80% identical in amino acid sequence to residues 31-346 of SEQ ID NO:2, wherein the sequence comprises cysteine residues corresponding to residues 58, 65, 132, 147, 153 and 219 of SEQ ID NO:2; and a transcription terminator, wherein the cell expresses the polypeptide encoded by the DNA segment.

Within another aspect is provided a method of producing a polypeptide comprising: culturing a cell into which has been introduced an expression vector comprising the following operably linked elements: a transcription promoter; a DNA segment encoding a polypeptide comprising a sequence of amino acid residues that is at least 80% identical in amino acid sequence to residues 31-346 of SEQ ID NO:2, wherein the sequence comprises cysteine residues corresponding to residues 58, 65, 132, 147, 153 and 219 of SEQ ID NO:2; and a transcription terminator; whereby the cell expresses the polypeptide encoded by the DNA segment; and recovering the expressed polypeptide.

Within still another aspect is provided a pharmaceutical composition comprising a polypeptide, the polypeptide comprising a sequence of amino acid residues that is at least 80% identical in amino acid sequence to residues 31-346 of SEQ ID NO:2, wherein the sequence comprises cysteine residues corresponding to residues 58, 65, 132, 147, 153 and 219 of SEQ ID NO:2; in combination with a pharmaceutically acceptable vehicle.

Within another aspect is provided an antibody that specifically binds to an epitope of a polypeptide comprising a sequence of amino acid residues that is at least 80% identical in amino acid sequence to residues 31-346 of SEQ ID NO:2, wherein the sequence comprises cysteine residues corresponding to residues 58, 65, 132, 147, 153 and 219 of SEQ ID NO:2.

Within yet another aspect is provided a binding protein that specifically binds to an epitope of a polypeptide comprising a sequence of amino acid residues that is at least 80% identical in amino the sequence to residues 31-346 of SEQ ID NO:2, wherein the sequence comprises cysteine residues corresponding to residues 58, 65, 132, 147, 153 and 219 of SEQ ID NO:2.

With still another aspect is provided an isolated polynucleotide encoding a polypeptide comprising a sequence of amino acid residues that is at least 80% identical in amino acid sequence to residues 31-346 of SEQ ID NO:2, wherein the sequence comprises cysteine residues corresponding to residues 58, 65, 132, 147, 153 and 219 of SEQ ID NO:2. Within one embodiment the polypeptide is at least 90% identical in amino acid sequence to residues 29-346 of SEQ ID NO:2, wherein the sequence comprises cysteine residues corresponding to residues 58, 65, 132, 147, 153 and 219 of SEQ ID NO:2. Within another embodiment the polypeptide comprises residues 1-346 of SEQ ID NO:2. Within another embodiment the polynucleotide is DNA.

Within another aspect is provided an isolated polynucleotide selected from the group consisting of: a) a sequence of nucleotides from nucleotide 47 to nucleotide 157 of SEQ ID NO:1; b) a sequence of nucleotides from nucleotide 131 to nucleotide 157 of SEQ ID NO:1; c) a sequence of nucleotides from nucleotide 137 to nucleotide 157 of SEQ ID NO:1; d) a sequence of nucleotides from nucleotide 164 to nucleotide 1084 of SEQ ID NO:1; e) a sequence of nucleotides from nucleotide 188 to nucleotide 1084 of SEQ ID NO:1; f) a sequence of nucleotides from nucleotide 131, 137 or 164 to nucleotide 181 of SEQ ID NO:1; g) degenerate nucleotide sequences of a), b), c), d), e) or f); and h) nucleotide sequences complementary to a), b), c), d), e), f) or g).

Within yet another embodiment is provided an isolated polynucleotide selected from the group consisting of: a) a sequence of nucleotides from nucleotide 131 to nucleotide 874 of SEQ ID NO:1; b) a sequence of nucleotide s from nucleotide 137 to nucleotide 874 of SEQ ID NO:1; c) a sequence of nucleotides from nucleotide 164 to nucleotide 874 of SEQ ID NO:1; d) a sequence of nucleotides from nucleotide 188 to nucleotide 874 of SEQ ID NO:1; e) a sequence of nucleotides from nucleotide 878 to nucleotide 1084 of SEQ ID NO:1; f) degenerate nucleotide sequences of a), b), c), d) or e) and g) nucleotide sequences complementary to a), b), c), d), e) or f).

Within another aspect is provided an isolated polynucleotide encoding a fusion protein consisting essentially of a first portion and a second portion joined by a peptide bond, the first portion comprises a polypeptide that is at least 80% identical in amino acid sequence to the amino acid sequence of a polypeptide selected from the group consisting of: a) a sequence of amino acid residues from amino acid residue 1, 29 or 31 to amino acid residue 37 of SEQ ID NO: 2; b) a sequence of amino acid residues from amino acid residue 40 or 48 to amino acid residue 346 of SEQ ID NO: 2; c) a sequence of amino acid residues from amino acid residue 29, 31 or 40 to amino acid residue 45 of SEQ ID NO: 2; d) a sequence of amino acid residues from amino acid residues 29, 31, 40 or 48 to amino acid residue 276 of SEQ ID NO:2; e) a sequence of amino acid residues from amino acid residue 278 to amino acid residue 346 of SEQ ID NO: 2; f) a sequence of amino acid residues from amino acid residue 1, 29 or 31 to amino acid residue 356 of SEQ ID NO:2; and g) a sequence of amino acid residues that is at least 80% identical in amino acid sequence to a), b), c), d) e) or f); and the second portion comprising another polypeptide.

Within another aspect is provided an isolated polynucleotide encoding a fusion protein comprising a secretory signal sequence having the amino acid sequence of amino acid residues 1-28 or 1-30 of SEQ ID NO:2, wherein the secretory signal sequence is operably linked to an additional polypeptide.

Within yet another aspect is provided an isolated polynucleotide comprising the sequence of nucleotide 1 to nucleotide 1084 of SEQ ID NO:13.

Within still another aspect is provided an oligonucleotide probe or primer comprising at least 14 contiguous nucleotides of a polynucleotide of SEQ ID NO:13 or a sequence complementary to SEQ ID NO:13.

Within another aspect is provided a method for detecting a genetic abnormality in a patient, comprising: obtaining a genetic sample from a patient; incubating the genetic sample with a polynucleotide comprising at least 14 contiguous nucleotides of SEQ ID NO:1 or the complement of SEQ ID NO:1, under conditions wherein the polynucleotide will hybridize to complementary polynucleotide sequence, to produce a first reaction product; comparing the first reaction product to a control reaction product, wherein a difference between the first reaction product and the control reaction product is indicative of a genetic abnormality in the patient.

### DETAILED DESCRIPTION OF THE INVENTION

Prior to setting forth the invention in detail, it may be helpful to the understanding thereof to define the following terms.

The term “affinity tag” is used herein to denote a peptide segment that can be attached to a polypeptide to provide for purification of the polypeptide or provide sites for attachment of the polypeptide to a substrate. In principal, any peptide or protein for which an antibody or other specific binding agent is available can be used as an affinity tag. Affinity tags include a polyhistidine tract, protein A (Nilsson et al., EMBO J. 4:1075, 1985; Nilsson et al., Methods Enzymol. 198:3, 1991), glutathione S transferase (Smith and Johnson, Gene 67:31, 1988), substance P, Flag™ peptide (Hopp et al., Biotechnology 6:1204-1210, 1988; available from Eastman Kodak Co., New Haven, Conn.), streptavidin binding peptide, or other antigenic epitope or binding domain. See, in general Ford et al., Protein Expression and Purification 2: 95-107, 1991. DNAs encoding affinity tags are available from commercial suppliers (e.g., Pharmacia Biotech, Piscataway, N.J.).

The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides and proteins. Where the context allows, these terms are used with reference to a particular sequence or portion of a polypeptide or protein to denote proximity or relative position. For example, a certain sequence positioned carboxyl-terminal to a reference sequence within a protein is located proximal to the carboxyl terminus of the reference sequence, but is not necessarily at the carboxyl terminus of the complete protein.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of polynucleotide molecules” denotes polynucleotide molecules having a complementary base sequence and reverse orientation as compared to a reference sequence. For example, the sequence 5′ATGCACGGG 3′ is complementary to 5′CCCGTGCAT 3′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” denotes a DNA molecule, linear or circular, that comprises a segment encoding a polypeptide of interest operably linked to additional segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both.

The term “isolated”, when applied to a polynucleotide molecule, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985). When applied to a protein, the term “isolated” indicates that the protein is found in a condition other than its native environment, such as apart from blood and animal tissue. In a preferred form, the isolated protein is substantially free of other proteins, particularly other proteins of animal origin. It is preferred to provide the protein in a highly purified form, i.e., greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, denotes that the segments are arranged so that they function in concert for their intended purposes, e.g. transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nt, more preferably 20-30 nt. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

The term “promoter” denotes a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. Most nuclear receptors also exhibit a multi-domain structure, including an amino-terminal, transactivating domain, a DNA binding domain and a ligand binding domain. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger peptide is commonly cleaved to remove the secretory peptide during transit through the secretory pathway.

A “soluble receptor” is a receptor polypeptide that is not bound to a cell membrane. Soluble receptors are most commonly ligand-binding receptor polypeptides that lack transmembrane and cytoplasmic domains. Soluble receptors can comprise additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis or translated from alternatively spliced mRNAs. Receptor polypeptides are said to be substantially free of transmembrane and intracellular polypeptide segments when they lack sufficient portions of these segments to provide membrane anchoring or signal transduction, respectively.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a secreted zsig46 polypeptide, wherein the DNA sequence is found on human chromosome 13. Potential N-glycosylation sites are located on the zsig46 polypeptides of the present invention at amino acids 118, 131, 166, 191, 243, 259, 269 and 340 of SEQ ID NO:2. Potential post-translational processing sites are the dibasic sites at amino acids 38-39 (RR), 46-47 (KR) and 277-278 (KR). Consequently, the polypeptides ranging from amino acid residue 40 (His) to 45 (Tyr), from 40 (His) to 276 (Ile) or from 48 (Phe) to 276 (Ile) may be released as an active polypeptide.

Analysis of the tissue distribution of the mRNA corresponding to this novel DNA showed that the polypeptide was expressed predominantly in thyroid. Weak expression was also observed in adrenal gland, trachea, lymph node, spinal cord, small intestine and ovary, however. Three transcript sizes were observed at approximately 1 kb, approximately 2 kb and approximately 3 kb. The polypeptide has been designated zsig46 polypeptide.

The novel zsig46 polypeptides of the present invention were initially identified by querying an EST database for secretory signal sequences, characterized by an upstream methionine start site, a hydrophobic region of approximately 13 amino acids and a cleavage site, in an effort to select for secreted proteins. Polypeptides corresponding to ESTs meeting those search criteria were compared to known sequences to identify secreted proteins having homology to known ligands. A single EST sequence was discovered and predicted to be a secreted protein; however, no homology to known proteins was identified. A search of databases accepting data from the human genome project revealed that the EST polynucleotide sequence was present on human chromosome 13. Further analysis of the genomic DNA revealed a polynucleotide encoding a full length secreted zsig46 polypeptide, wherein the polynucleotide was characterized by two introns. A deletion was observed in the EST sequence, and a “C” was inserted to replace the EST-deletion. Zsig46 polypeptide is encoded, in part, in a human chromosome segment DNA clone, Genbank locus number HSAC001226, from position 23544 to 27536. Two introns were predicted at base positions 23681-26472 and 26636-27310 of human chromosome 13 (clone HSAC001226) as submitted by Hawkins et al. Intron/exon boundaries were predicted based on a comparison of the sequence of zsig46 mRNA with the genomic sequence. The sequence of zsig46 was confirmed as described in Example 1 and the confirmed sequence is shown in SEQ ID NO: 1.

Zsig46 polypeptides, ranging from amino acid 1 (Met) to amino acid 346 (Leu); the alternative mature zsig46 polypeptides, ranging from amino acid 29(Trp) or amino acid 31 (Arg) to amino acid 346 (Leu); or the alternative secretion leader fragments thereof, which fragments range from amino acid 1 (Met) to amino acid 28 (Gly) or amino acid 30 (Ser) may be used in the study of secretion of proteins from cells. In preferred embodiments of this aspect of the present invention, the mature polypeptides are formed as fusion proteins with putative secretory signal sequences; plasmids bearing regulatory regions capable of directing the expression of the fusion protein is introduced into test cells; and secretion of mature protein is monitored. In other preferred embodiments of this aspect of the present invention, the alternative secretion leader fragments are formed as fusion proteins with alternative proteins selected for secretion; plasmids bearing regulatory regions capable of directing the expression of the fusion protein are introduced into test cells; and secretion of the protein is monitored. Such secretion may be monitored by running gels on the supernatant or contacting the supernatant with zsig46-specific antibodies. Both of these techniques are well known in the art.

The present invention also encompasses fragments of the zsig46 polypeptide. Potential post-translational processing sites are found at amino acids 38-39 (RR), 46-47 (KR) and 277-278 (KR) of SEQ ID NO: 2. Thus, fragments of the zsig46 polypeptides of the present invention include those polypeptides encompassing (a) amino acid residue 1 (Met), 29 (Trp) or 31 (Arg) to amino acid residue 37 (Ser) of SEQ ID NO: 2; (b) amino acid residue 40 (His) or 48 (Phe) to amino acid residue 346 (Leu) of SEQ ID NO: 2; (c) amino acid residue 40 (His) to amino acid residue 45 (Tyr) of SEQ ID NO: 2; (d) amino acid residue 29 (Trp), 31 (Arg), 40 (His) or 48 (Phe) to amino acid residue 276 (Ile); and (e) amino acid residue 278 (Phe) to amino acid residue 346 (Leu) of SEQ ID NO: 2. Polypeptides sharing at least 80% identity with the zsig46 polypeptide fragments are also contemplated by the present invention.

Polynucleotides encoding such fragments are also encompassed by the present invention, including the group consisting of (a) polynucleotide molecules comprising a sequence of nucleotides as shown in SEQ ID NO: 1 from nucleotide 1, 47, 131 or 136 to nucleotide 157; (b) polynucleotide molecules comprising a sequence of nucleotides as shown in SEQ ID NO: 1 from nucleotide 164 or 188 to nucleotide 1084; (c) polynucleotide molecules comprising a sequence of nucleotides as shown in SEQ ID NO: 1 from nucleotide 164 to nucleotide 181 or 1084; (d) polynucleotide molecules comprising a sequence of nucleotides as shown in SEQ ID NO: 1 from nucleotide 130, 136, 164 or 168 to nucleotide 872; (e) polynucleotide molecules comprising a sequence of nucleotides as shown in SEQ ID NO: 1 from nucleotide 878 to nucleotide 1084. The present invention also contemplates degenerate polynucleotide molecules encoding a zsig46 polypeptide fragment. Polynucleotide molecules encoding polypeptides sharing at least 80% identity with the zsig46 polypeptide fragments are also contemplated by the present invention.

The present invention also provides polynucleotide molecules, including DNA and RNA molecules, that encode the zsig46 polypeptides disclosed herein. Those skilled in the art will readily recognize that, in view of the degeneracy of the genetic code, considerable sequence variation is possible among these polynucleotide molecules. SEQ ID NO:13 is a degenerate DNA sequence that encompasses all DNAs that encode the zsig46 polypeptide of SEQ ID NO:2. Those skilled in the art will recognize that the degenerate sequence of SEQ ID NO:13 also provides all RNA sequences encoding SEQ ID NO:2 by substituting U (uracil) for T (thymine). Thus, zsig46 polypeptide-encoding polynucleotides comprising nucleotide 1 to nucleotide 1084 of SEQ ID NO:13 and their RNA equivalents are contemplated by the present invention. Table 1 sets forth the one-letter codes used within SEQ ID NO:13 to denote degenerate nucleotide positions. “Resolutions” are the nucleotides denoted by a code letter. “Complement” indicates the code for the complementary nucleotide(s). For example, the code Y denotes either C (cytosine) or T, and its complement R denotes A (adenine) or G (guanine), A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:13, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:13 serves as a template for optimizing expression of polynucleotides in various cell types and species commonly used in the art and disclosed herein. Sequences containing preferential codons can be tested and optimized for expression in various species, and tested for functionality as disclosed herein.

The present invention also provides post-translationally modified polypeptides or polypeptide fragments. Examples of post translational modifications include proteolytic cleavage, glycosylation, disulfide bonding and hydroxylation. Many proteins are cleaved or processed into active form through the action of prohormone convertases (endoproteases). The most prevalent cleavage or processing site is a dibasic amino acid prohormone convertase site. There are only a few dibasic amino acid combinations, including Lys-Lys, Arg-Arg, Arg-Lys and Lys-Arg. Non-dibasic cleavage and processing sites have also been observed, for example, Asn-Arg is a non-dibasic site found in gastrin. Potential dibasic sites located on the zsig46 polypeptides of the present invention include amino acids 38-39 (Arg-Arg), 46-47 (Lys-Arg) and 277-278 (Lys-Arg). Zsig46 polypeptides may be processed by prohormone convertases into an active from. Known prohormone convertases include, but are not limited to, prohormone convertase 3 (PC3), prohormone convertase 2 (PC2), furin, or similar convertases of the furin family such as prohormone convertase 4 (PC4) and PACE4. Consequently, the polypeptides ranging from amino acid residue 1, 29 or 31 to residue 37, from residue 1, 29, 31 or 40 to residue 45, from residue 1, 29, 31, 40 or 48 to residue 276 or from residue 40, 49 or 279 to residue 346, may be released as an active polypeptide. Additional post-translational sites located on the zsig46 polypeptides of the present invention include potential N-glycosylation sites at amino acids 118, 131, 166, 191, 243, 259, 269 and 340 of SEQ ID NO:2.

The present invention also provides zsig46 fusion proteins. For example, fusion proteins of the present invention encompass (1) a polypeptide selected from the following: polypeptides encompassing (a) amino acid residue 1 (Met), 29 (Trp) or 31 (Arg) to amino acid residue 37 (Ser) of SEQ ID NO: 2; (b) amino acid residue 40 (His) or 48 (Phe) to amino acid residue 346 (Leu) of SEQ ID NO: 2; (c) amino acid residue 40 (His) to amino acid residue 45 (Tyr) or 346 (Leu) of SEQ ID NO: 2; (d) amino acid residue 29 (Trp), 31 (Arg), 40 (His) or 48 (Phe) to amino acid residue 276 (Ile); and (e) amino acid residue 278 (Phe) to amino acid residue 346 (Leu) of SEQ ID NO: 2; and (2) another polypeptide. The other polypeptide may be a signal peptide to facilitate secretion of the fusion protein, a therapeutically active protein, a targeting protein or the like. Other fusion proteins of the present invention comprise (1) a polypeptide including amino acid residues 40 (His) to 45 (Tyr), flanked by alternative processing sites, such as dibasic sites and (2) another polypeptide.

The present invention also encompasses dimers of zsig46 polypeptides. Zsig46 polypeptides include six cysteine residues at positions 58, 65, 132, 147, 153, 219 and of SEQ ID NO:2 in the mature polypeptide and at position 15 of SEQ ID NO:2 in the putative signal sequence. The presence of these cysteines suggest that covalent disulfide bonds between zsig46 monomers may be formed. The cysteine residues may also be indicative of up to three internal disulfide bonds in the zsig46 polypeptide structure. A combination zsig46 monomer-zsig46 monomer disulfide bonds and zsig46 polypeptide internal disulfide bonds may also be formed. Such covalent disulfide can also form between zsig46 and other polypeptides having compatible cysteine residues. Thus the invention contemplates homodimer and heterodimer zsig46 complexes formed by intermolecular disulfide bonds formed between cysteine residues. The invention provides an isolated protein comprising a first polypeptide that is at least 80% identical in amino acid sequence to the amino acid sequence of a polypeptide selected from the group consisting of: a) amino acid residues 1-29 or 30 of SEQ ID NO:2, said polypeptide comprising a cysteine residue at a position corresponding to residue 15 of SEQ ID NO:2; b) amino acid residues 48-276 of SEQ ID NO:2, the polypeptide comprising cysteine residues at positions corresponding to residues 58, 65, 132, 147, 153 and 219 of SEQ ID NO:2; and c) amino acid residues 31-346 of SEQ ID NO:2, the polypeptide comprising cysteine residues at positions corresponding to residues 58, 65, 132, 147, 153 and 219 of SEQ ID NO:2; complexed to a second polypeptide. Suitable second polypeptides include zsig46 polypeptides and other polypeptides having compatible cysteine residues.

The highly conserved amino acids within a protein family can be used as a tool to identify new family members. For instance, reverse transcription-polymerase chain reaction (RT-PCR) can be used to amplify sequences encoding the conserved motifs from RNA obtained from a variety of tissue sources. In particular, highly degenerate primers designed from conserved sequences are useful for this purpose. In particular, the following primers are useful for this purpose:

Amino acids 90-95 of SEQ ID NO: 2 (corresponding to nucleotides 314-331 of SEQ ID NO: 1);

Amino acids 119-124 of SEQ ID NO: 2 (corresponding to nucleotides 401-418 of SEQ ID NO: 1);

Amino acids 145-150 of SEQ ID NO: 2 (corresponding to nucleotides 479-496 of SEQ ID NO: 1);

Amino acids 162-167 of SEQ ID NO: 2 (corresponding to nucleotides 530-547 of SEQ ID NO: 1); and

Amino acids 179-184 of SEQ ID NO: 2 (corresponding to nucleotides 581-598 of SEQ ID NO: 1).

The present invention further provides pharmaceutical compositions comprising purified zsig46 polypeptide in combination with a pharmaceutically acceptable vehicle. Such pharmaceutical compositions would be useful in treatment or prevention of deficiencies in expression of secreted proteins encoded by human chromosome 13. More specifically, the pharmaceutical compositions may find use in the prevention or treatment of conditions associated with chromosome 13q. Examples of diseases of the nervous system associated with this region include Hirschsprung's disease, neuronal ceroid-lipofucinosis, Wilson disease, Reiger syndrome or the like or conditions associated with pathological over or under expression of BRN3A Pou-Domain transcription factor or serotonin 5-HT-2 receptor. Northern analysis indicates that zsig46 is predominantly expressed in thyroid. Consequently, zsig46 polypeptide may be useful in treatment or prevention of thyroid diseases. Examples of diseases associated with the thyroid include sick erythroid syndrome, hypothyroidism, Grave's disease, thyrotoxicosis, thyroid cancers or the like.

More specifically, the zsig46 polypeptide-encoding gene is located in close proximity to the endothelin-B receptor gene, which is one of the 13-linked susceptibility gene for Hirschsprung disease. Hirschsprung disease is a multigenic disorder that involves a defect in the development of neural crest-derived cell lineages. See, for example, Puffenberger et al., Cell 79: 1257-66, 1994. An association between Hirschsprung disease and cancer of the thyroid has also been observed in some cases. See, for example, Rakeover et al., Journal of Pediatric Endocrinoloqy 7(4): 373-7, 1994. This association may be explained by the fact that cell types involved in both conditions (C-cells of the thyroid and intestinal enterocromaffin cells from the neural crest) have a common origin. The piebald-lethal mutations in mice are believed to mimic human Hirschsprung disease. See, for example, Hosoda et al., Cell 79: 1267-76, 1994.

Neuronal ceroid-lipofuscinosis maps to 13q21-q32 and is characterized by psychomotor deterioration, visual failure and accumulation of autofluoroescent lipopigment in neurons or other cell types. See, for example, Savukoski et al., Am. J. Hum. Genet. 55: 695-701, 1994 and Klockars et al., Genomics 35: 71-8, 1996.

Wilson disease maps to 13q14-q21 and is associated with changes in basal ganglia and liver. These changes typically involve neurological manifestations and signs of cirrhosis. See, for example, Tanzi et al, Am. J. Hum. Genet. 53(Suppl.): A228, 1993 and Tanzi et al., Nature Genet. 5: 344-50, 1993. The LEC (Long-Evans Cinnamon) rat has been found to be an authentic model of Wilson disease. See, for example, Li et al., J. Clin. Invest. 87: 1858-61, 1991.

Reiger syndrome maps to 13q14 and is associated with abnormal development of anterior eye segment, tooth enamel and other structures associated with the embryonic neural crest. See, for example, Phillips et al., Am. J. Hum. Genet. 59: 613-619, 1996.

The BRN3A Pou-Domain transcription factor-encoding gene maps to 13q21-q32. This protein is highly expressed in developing sensory nervous system and in cells of B- and T-lymphocytic lineage and is involved in the growth and differentiation of neurons. See, for example, Still and Cowell, Cytogenet. Cell Genet. 74 225-6, 1996. Consequently, pathological expression of this gene is likely to be associated with abnormal neuron growth and/or differentiation.

The serotonin 5-HT-2 receptor-encoding gene maps to 13q14-q21. This receptor is a neurotransmitter implicated in sleep, appetite, pain perception, hormone secretion, sexual behavior and the like. See, for example, Sparkes et al., Genomics 9: 461-5, 1991. Consequently, pathological expression of this gene is likely to be associated with disruptions in one or several of those important processes, such as mental depression, migraine, epilepsy, obsessive-compulsive behavior, affective disorder or the like.

Cell lines which can be used to evaluate zsig46 polypeptide, fragments thereof, fusion proteins containing the same, antibodies, agonists and antagonists with respect to diagnostic, prophylactic or treatment of thyroid conditions are those which are known and available. Exemplary thyroid cell lines include the following: MTC SK (human carcinoma; Pfragner et al., Cancer Research 50: 4160-66, 1990); FRT (rat thyroid; Ambesi-Impiombato & Coon, Int. Rev. Cytol. Suppl. 10: 163-71, 1979); 8505C (human carcinoma; Ito et al., Int. J. Oncol. 4: 583-6, 1994); 8305C (human carcinoma, undifferentiated; Ito et al. referenced above); FTC-238 (human carcinoma; Packman et al., Surgery 118(6): 1011-6, 1995); S117 (human sarcoma; Wiedemann et al., J. Cancer Res. Clin. Oncol. 118(2): 129-35, 1992); and Bthy.ts1 (human thyroid, follicular; Robbins & Scharff, J. Cell Biol. 34: 684-6, 1967).

Radiation hybrid mapping is a somatic cell genetic technique developed for constructing high-resolution, contiguous maps of mammalian chromosomes (Cox et al., Science 250:245-250, 1990). Partial or full knowledge of a gene's sequence allows the designing of PCR primers suitable for use with chromosomal radiation hybrid mapping panels. Commercially available radiation hybrid mapping panels which cover the entire human genome, such as the Stanford G3 RH Panel and the GeneBridge 4 RH Panel (Research Genetics, Inc., Huntsville, Ala.), are available. These panels enable rapid, PCR based, chromosomal localizations and ordering of genes, sequence-tagged sites (STSs), and other nonpolymorphic- and polymorphic markers within a region of interest. This includes establishing directly proportional physical distances between newly discovered genes of interest and previously mapped markers. The precise knowledge of a gene's position can be useful in a number of ways including: 1) determining if a sequence is part of an existing contig and obtaining additional surrounding genetic sequences in various forms such as YAC-, BAC- or cDNA clones, 2) providing a possible candidate gene for an inheritable disease which shows linkage to the same chromosomal region, and 3) for cross-referencing model organisms such as mouse which may be beneficial in helping to determine what function a particular gene might have.

The results showed that the zsig46 gene maps 217.1 CR_{—}3000 from the top of the human chromosome 13 linkage group on the WICGR radiation hybrid map. Proximal and distal framework markers were AFM350XA5 and CHLC.GATA30F02 (D13S789), respectively. The use of surrounding markers positions Zsig46 in the 13q22.2-q22.3 region on the integrated LDB chromosome 13 map.

The zsig46 polypeptide-encoding gene is located on chromosome 13 in the vicinity of the RNHA_HUMAN gene, as described by Lee and Hurwitz, J. Biol. Chem. 268(22): 16822-30, 1993. This gene encodes RNA helicase A, an abundant nuclear enzyme of HeLa cells that unwinds double-stranded RNA in a 3′ to 5′ direction. RNA helicase A is a 1279 amino acid member of the DEAH protein family of RNA-dependent ATPases and/or RNA helicases. RNA helicase A is postulated to play a role in X-linked gene expression in both human sexes.

If a mammal has a mutated or lacks a zsig46 gene, the zsig46 gene can be introduced into the cells of the mammal. In one embodiment, a gene encoding a zsig46 polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector (Kaplitt et al., Molec. Cell. Neurosci. 2:320-330 (1991)), an attenuated adenovirus vector, such as the vector described by Stratford-Perricaudet et al., J. Clin. Invest. 90:626-630 (1992), and a defective adeno-associated virus vector (Samulski et al., J. Virol. 61:3096-3101 (1987); Samulski et al. J. Virol. 63:3822-3828 (1989)).

In another embodiment, the gene can be introduced in a retroviral vector, e.g., as described in Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol., 62:1120 (1988); Temin et al., U.S. Pat. No. 5,124,263; International Patent Publication No. WO 95/07358, published Mar. 16, 1995 by Dougherty et al.; and Blood, 82:845 (1993).

Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA, 84:7413-7417 (1987); see Mackey et al., Proc. Natl. Acad. Sci. USA, 85:8027-8031 (1988)). The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

It is possible to remove the cells from the body and introduce the vector as a naked DNA plasmid and then re-implant the transformed cells into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem., 267:963-967 (1992); Wu et al., J. Biol. Chem., 263:14621-14624 (1988); and Johnston and Tang, Methods in Cell Biology 43: 353-65 (1994).

Another aspect of the present invention involves antisense polynucleotide compositions that are complementary to a segment of the polynucleotides set forth in SEQ ID NO: 1. Such synthetic antisense oligonucleotides are designed to bind to mRNA encoding zsig46 polypeptides and inhibit translation of such mRNA. Such antisense oligonucleotides are useful to inhibit expression of zsig46 polypeptide-encoding genes in cell culture or in a subject.

The present invention also provides reagents which will find use in diagnostic applications. For example, the zsig46 gene, a probe comprising zsig46 DNA or RNA or a subsequence thereof can be used to determine if the zsig46 gene is present on chromosome 13 or if a mutation has occurred. Detectable chromosomal aberrations at the zsig46 gene locus include but are not limited to aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel, et. al., ibid.; Marian, A. J., Chest, 108: 255-265, 1995).

The present invention also provides reagents which will find use in diagnostic applications. For example, the zsig46 gene, a probe comprising zsig46 DNA or RNA, or a subsequence thereof can be used to determine if the zsig46 gene is present on chromosome 17 or if a mutation has occurred. Detectable chromosomal aberrations at the zsig46 gene locus include, but are not limited to, aneuploidy, gene copy number changes, insertions, deletions, restriction site changes and rearrangements. These aberrations can occur within the coding sequence, within introns, or within flanking sequences, including upstream promoter and regulatory regions, and may be manifested as physical alterations within a coding sequence or changes in gene expression level.

In general, these diagnostic methods comprise the steps of (a) obtaining a genetic sample from a patient; (b) incubating the genetic sample with a polynucleotide probe or primer as disclosed above, under conditions wherein the polynucleotide will hybridize to complementary polynucleotide sequence, to produce a first reaction product; and (iii) comparing the first reaction product to a control reaction product. A difference between the first reaction product and the control reaction product is indicative of a genetic abnormality in the patient. Genetic samples for use within the present invention include genomic DNA, cDNA, and RNA. The polynucleotide probe or primer can be RNA or DNA, and will comprise a portion of SEQ ID NO:1, the complement of SEQ ID NO:1, or an RNA equivalent thereof. Suitable assay methods in this regard include molecular genetic techniques known to those in the art, such as restriction fragment length polymorphism (RFLP) analysis, short tandem repeat (STR) analysis employing PCR techniques, ligation chain reaction (Barany, PCR Methods and Applications 1:5-16, 1991), ribonuclease protection assays, and other genetic linkage analysis techniques known in the art (Sambrook et al., ibid.; Ausubel et. al., ibid.; Marian, Chest 108:255-65, 1995). Ribonuclease protection assays (see, e.g., Ausubel et al., ibid., ch. 4) comprise the hybridization of an RNA probe to a patient RNA sample, after which the reaction product (RNA-RNA hybrid) is exposed to RNase. Hybridized regions of the RNA are protected from digestion. Within PCR assays, a patient's genetic sample is incubated with a pair of polynucleotide primers, and the region between the primers is amplified and recovered. Changes in size or amount of recovered product are indicative of mutations in the patient. Another PCR-based technique that can be employed is single strand conformational polymorphism (SSCP) analysis (Hayashi, PCR Methods and Applications 1:34-8, 1991).

Another aspect of the present invention involves the detection of zsig46 polypeptides in a cell culture of tumor cells or in a serum sample or tissue biopsy of a patient undergoing evaluation for thyroid dysfunction. Zsig46 polypeptides can be detected using immunoassay techniques and antibodies capable of recognizing a zsig46 polypeptide epitope. More specifically, the present invention contemplates methods for detecting zsig46 polypeptide comprising:

exposing a solution or sample or cell culture lysate or supernatant, possibly containing zsig46 polypeptide, to an antibody attached to a solid support, wherein said antibody binds to a first epitope of a zsig46 polypeptide;

washing said immobilized antibody-polypeptide to remove unbound contaminants;

exposing the immobilized antibody-polypeptide to a second antibody directed to a second epitope of a zsig46 polypeptide, wherein the second antibody is associated with a detectable label; and

detecting the detectable label. Zsig46 polypeptide concentration differing from that of controls may be indicative of dysfunction of the thyroid. Examples of dysfunction of the thyroid include sick eurthyroid syndrome, hypothyroidism, Grave's disease, thyrotoxicosis, thyroid cancers or the like.

Within additional aspects of the invention there are provided antibodies that specifically bind to the zsig46 polypeptides described above. Such antibodies are useful for, among other uses as described herein, preparation of anti-idiotypic antibodies. An additional aspect of the present invention provides methods for identifying agonists or antagonists of the zsig46 polypeptides disclosed above, which agonists or antagonists may have valuable properties as discussed further herein. Within one embodiment, there is provided a method of identifying zsig46 polypeptide agonists, comprising providing cells responsive thereto, culturing the cells in the presence of a test compound and comparing the cellular response with the cell cultured in the presence of the zsig46 polypeptide, and selecting the test compounds for which the cellular response is of the same type.

Within another embodiment, there is provided a method of identifying antagonists of zsig46 polypeptide, comprising providing cells responsive to a zsig46 polypeptide, culturing a first portion of the cells in the presence of zsig46 polypeptide, culturing a second portion of the cells in the presence of the zsig46 polypeptide and a test compound, and detecting a decrease in a cellular response of the second portion of the cells as compared to the first portion of the cells. Other techniques for antagonist, agonist or antibody identification, such as phage display, may also be used.

Molecules of the present invention can be used to identify and isolate receptors involved in zsig46 binding and signaling processes. For example, proteins or peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-737) or photoaffinity labeled (Brunner et al., Ann. Rev. Biochem. 62:483-514, 1993 and Fedan et al., Biochem. Pharmacol. 33:1167-1180, 1984) and specific cell-surface proteins can be identified. In addition to membrane preparations, putative soluble receptors or putative membrane-bearing cells may also be used. Moreover, in situ ligand binding techniques may be used to identify cell-associated and soluble zsig46 polypeptide receptors.

Transgenic mice, engineered to express the zsig46 gene, and mice that exhibit a complete absence of zsig46 gene function, referred to as “knockout mice” (Snouwaert et al., Science 257:1083, 1992), may also be generated (Lowell et al., Nature 366:740-742, 1993). These mice may be employed to study the zsig46 gene and the protein encoded thereby in an in vivo system.

Within preferred embodiments of the invention the isolated polynucleotides will hybridize to similar sized regions of SEQ ID NO:2, SEQ ID NO:5, SEQ ID NO:6, other probes specifically recited herein, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is up to about 0.03 M pH 7 and the temperature is at least about 60° C.

As previously noted, the isolated polynucleotides of the present invention include DNA and RNA. Methods for isolating DNA and RNA are well known in the art. It is generally preferred to isolate RNA from thyroid, pituitary, kidney, brain, intestine, colon, tumor tissue of various types, although DNA can also be prepared using RNA from other tissues or isolated as genomic DNA. Total RNA can be prepared using guanidine HCl extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-1412, 1972). Complementary DNA (cDNA) is prepared from poly(A)⁺ RNA using known methods. Polynucleotides encoding zsig46 polypeptides are then identified and isolated by, for example, hybridization or PCR.

The present invention further provides counterpart polypeptides and polynucleotides from other species (orthologs). These species include, but are not limited to mammalian, avian, amphibian, reptile, fish, insect and other vertebrate and invertebrate species. Of particular interest are zsig46 polypeptides from other mammalian species, including murine, rat, porcine, ovine, bovine, canine, feline, equine and other primate proteins. Orthologs of the human proteins can be cloned using information and compositions provided by the present invention in combination with conventional cloning techniques. For example, a cDNA can be cloned using mRNA obtained from a tissue or cell type that expresses the protein. Suitable sources of mRNA can be identified by probing Northern blots with probes designed from the sequences disclosed herein. A library is then prepared from mRNA of a positive tissue of cell line. A zsig46-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the sequences disclosed herein. Within an additional method, the cDNA library can be used to transform or transfect host cells, and expression of the cDNA of interest can be detected with an antibody to zsig46 polypeptide. Similar techniques can also be applied to the isolation of genomic clones.

Those skilled in the art will recognize that the sequences disclosed in SEQ ID NO:1 and SEQ ID NO:2 represent a single allele of the human zsig46 gene and polypeptide, and that allelic variation and alternative splicing, “splice variants”, are expected to occur. Allelic variants can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the zsig46 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated zsig46 polypeptides that are substantially homologous to the polypeptides of SEQ ID NO:2 and their orthologs. The term “substantially homologous” is used herein to denote polypeptides having 50%, preferably 60%, more preferably at least 80%, sequence identity to the sequences shown in SEQ ID NO:2 or their orthologs. Such polypeptides will more preferably be at least 90% identical, and most preferably 95% or more identical to SEQ ID NO:2 or its orthologs. Percent sequence identity is determined by conventional methods. See, for example, Altschul et al., Bull. Math. Bio. 48: 603-616, 1986 and Henikoff and Henikoff, Proc. Natl. Acad. Sci. USA 89:10915-10919, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\begin{matrix}[ length of the longer sequence plus the number of gaps \\ introduced into the longer sequence in order to \\ align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Substantially homologous proteins and polypeptides are characterized as having one or more amino acid substitutions, deletions or additions. These changes are preferably of a minor nature, that is conservative amino acid substitutions (see Table 4) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or an affinity tag. Polypeptides comprising affinity tags can further comprise a proteolytic cleavage site between the zsig46 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The proteins of the present invention can also comprise non-naturally occurring amino acid residues. Non-naturally occurring amino acids include, without limitation, trans-3-methylproline, 2,4-methanoproline, cis-4-hydroxy-proline, trans-4-hydroxyproline, N-methyl-glycine, allo-threonine, methylthreonine, hydroxy-ethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zsig46 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity (e.g., secretion, receptor-binding or the like) as disclosed below to identify amino acid residues that are critical to the activity of the molecule. See also, Hilton et al., J. Biol. Chem. 271:4699-708, 1996. Sites of ligand-receptor or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related polypeptides.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zsig46 DNA and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed above can be combined with high-throughput, automated screening methods to detect activity of cloned, mutagenized polypeptides in host cells. Mutagenized DNA molecules that encode active polypeptides (e.g., those capable of secretion, receptor binding or the like) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can identify and/or prepare a variety of polypeptides that are substantially homologous to residues 29 (Trp) or 31 (Arg) to 346 (Leu) of SEQ ID NO: 2 or allelic variants thereof and retain the secretion, receptor binding or like properties of the wild-type protein. Such polypeptides may include additional amino acids from affinity tags or the like. Such polypeptides may also include additional polypeptide segments as generally disclosed above.

The polypeptides of the present invention, including full-length proteins, fragments thereof and fusion proteins, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al. (eds.), Current Protocols in Molecular Biology, John Wiley and Sons, Inc., NY, 1987.

In general, a DNA sequence encoding a zsig46 polypeptide of the present invention is operably linked to other genetic elements required for its expression, generally including a transcription promoter and terminator within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zsig46 polypeptide into the secretory pathway of a host cell, a secretory signal sequence (also known as a leader sequence, prepro sequence or pre sequence) is provided in the expression vector. The secretory signal sequence may be that of the zsig46 polypeptide, or may be derived from another secreted protein (e.g., t-PA) or synthesized de novo. The secretory signal sequence is joined to the zsig46 polypeptide-encoding DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Conversely, the signal sequence portion of the zsig32 polypeptide (amino acids 1-28 or 1-30 of SEQ ID NO. 2) may be employed to direct the secretion of an alternative protein by analogous methods. A signal fusion polypeptide can be made wherein a secretory signal sequence derived from amino acid residues 1-28 or 1-30 of SEQ ID NO:2 is operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein, such as a receptor. Such fusions may be used in vivo or in vitro to direct peptides through the secretory pathway.

Cultured mammalian cells are also suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981; Graham and Van der Eb, Viroloqy 52:456, 1973), electroporation (Neumann et al., EMBO J. 1:841-845, 1982); DEAE-dextran mediated transfection (Ausubel et al., eds., Current Protocols in Molecular Biology, John Wiley and Sons, Inc., NY, 1987), liposome-mediated transfection (Hawley-Nelson et al., Focus 15:73, 1993; Ciccarone et al., Focus 15:80, 1993), and viral vectors (A. Miller and G. Rosman, BioTechniques 7:980-90, 1989; Q. Wang and M. Finer, Nature Med. 2:714-16, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

If the zsig46 polypeptide is expressed in a non-endocrine or non-neuroendocrine cell, the expression host cell generally will not express the prohormone convertases PC2 and PC3, which are believed to be involved in the regulated secretory pathway. Another member of this endoprotease family, furin, is present in most cells and is believed to be involved in the constitutive secretory pathway. Vollenweider et al. have described the role of these prohormone conversion endoproteases in general, and specifically describe studies involving co-transfection of COS cells with proinsulin and one of the endoproteases (Diabetes 44:1075-80, 1995). Their results showed that PC3 and furin were able to cleave proinsulin at both its junctions; PC2 did not exhibit prohormone cleavage to any significant extent. Without co-transfection of an endoprotease, the prohormone was not converted to any great extent by COS cells. However, the co-transfection system described is still not an exact model of the natural β cell environment, since β cells make both PC2 and PC3. Also, a non-endocrine cell does not represent a native environment for PC2 and PC3 expression. In addition, co-transfection may result in general or local overexpression of PC2 and/or PC3, relative to the native β cell environment. In a preferred embodiment, the host cells will be co-transfected with a second DNA expression construct comprising the following operably linked elements: a transcription promoter; a DNA segment encoding an endoprotease; and a transcription terminator, wherein the host cell expresses the DNA segment encoding the endoprotease.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King and Possee, The Baculovirus Expression System: A Laboratory Guide, London, Chapman & Hall; O'Reilly et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant zsig46 baculovirus utilizes a transposon-based system described by Luckow (Luckow et al., J. Virol. 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the zsig46 polypeptide into a baculovirus genome maintained in E. coli as a large plasmid called a “bacmid.” See, Hill-Perkins and Possee, J. Gen. Virol. 71:971-6, 1990; Bonning et al., J. Gen. Virol. 75:1551-6, 1994; and, Chazenbalk and Rapoport, J. Biol. Chem. 270:1543-9, 1995. In addition, transfer vectors can include an in-frame fusion with DNA encoding an epitope tag at the C- or N-terminus of the expressed zsig46 polypeptide, for example, a Glu-Glu epitope tag (Grussenmeyer et al., Proc. Natl. Acad. Sci. 82:7952-4, 1985). Using a technique known in the art, a transfer vector containing zsig46 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zsig46 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King and Possee, ibid.; O'Reilly et al., ibid.; Richardson, ibid.). Subsequent purification of the zsig46 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. Nos. 4,599,311; Kawasaki et al., 4,931,373; Brake, 4,870,008; Welch et al., 5,037,743; and Murray et al., 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. Nos. 4,615,974; and Bitter, 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (τ) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zsig46 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Expressed recombinant zsig46 polypeptides (or chimeric zsig46 polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable anion exchange media include derivatized dextrans, agarose, cellulose, polyacrylamide, specialty silicas, and the like. PEI, DEAE, QAE and Q derivatives are preferred, with DEAE Fast-Flow Sepharose (Pharmacia, Piscataway, N.J.) being particularly preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their structural properties. For example, immobilized metal ion adsorption (IMAC) chromatography can be used to purify histidine-rich proteins, or proteins having a His-tag. Briefly, a gel is first charged with divalent metal ions to form a chelate (E. Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional preferred embodiments of the invention, a fusion of the polypeptide of interest and an affinity tag (e.g., maltose-binding protein, FLAG, Glu-Glu, an immunoglobulin domain) may be constructed to facilitate purification as is discussed in greater detail in the Example sections below.

Protein refolding (and optionally reoxidation) procedures may be advantageously used. It is preferred to purify the protein to >80% purity, more preferably to >90% purity, even more preferably >95%, and particularly preferred is a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

Zsig46 polypeptides or fragments thereof may also be prepared through chemical synthesis. Zsig46 polypeptides may be monomers or multimers; glycosylated or non-glycosylated; pegylated or non-pegylated; and may or may not include an initial methionine amino acid residue.

A zsig46 ligand-binding polypeptide can also be used for purification of ligand. The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the receptor polypeptide. The ligand is then eluted using changes in salt concentration, chaotropic agents (guanidine HCl), or pH to disrupt ligand-receptor binding.

An assay system that uses a ligand-binding receptor (or an antibody, one member of a complement/anti-complement pair) or a binding fragment thereof, and a commercially available biosensor instrument (BIAcore™, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

An in vivo approach for assaying proteins of the present invention involves viral delivery systems. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161-89, 1994; and Douglas and Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293S cells can be grown in suspension culture at relatively high cell density to produce significant amounts of protein (see Garnier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

Additionally, purified proteins of the present invention can be assayed in vivo following administration by intravenous injection of the protein into a suitable animal model. Mice receiving daily injections with purified zsig46 showed a statistically significant decrease in platelet count when compared to control mice. They also exhibited minor changes in clinical chemistry, including elevated creatinine and phosphorous levels.

Zsig46 polypeptides can also be used to prepare antibodies that specifically bind to zsig46 epitopes, peptides or polypeptides. Methods for preparing polyclonal and monoclonal antibodies are well known in the art (see, for example, Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, hamsters, guinea pigs and rats as well as transgenic animals such as transgenic sheep, cows, goats or pigs. Antibodies may also be expressed in yeast and fungi in modified forms as well as in mammalian and insect cells. The zsig46 polypeptide or a fragment thereof serves as an antigen (immunogen) to inoculate an animal or elicit an immune response. Suitable antigens would include the zsig46 polypeptide encoded by SEQ ID NO:2 from amino acid residue 29-34G of SEQ ID NO:2, from amino acid residue 31-346 of SEQ ID NO:2, or a contiguous 9-346 amino acid residue fragment thereof. The immunogenicity of a zsig46 polypeptide may be increased through the use of an adjuvant, such as alum (aluminum hydroxide) or Freund's complete or incomplete adjuvant. Polypeptides useful for immunization also include fusion polypeptides, such as fusions of zsig46 or a portion thereof with an immunoglobulin polypeptide or with an affinity tag. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting only non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Alternative techniques for generating or selecting antibodies useful herein include in vitro exposure of lymphocytes to zsig46 protein or peptide, and selection of antibody display libraries in phage or similar vectors (for instance, through use of immobilized or labeled zsig46 protein or peptide).

Antibodies are defined to be specifically binding if: 1) they exhibit a threshold level of binding activity, and/or 2) they do not significantly cross-react with related polypeptide molecules. First, antibodies herein specifically bind if they bind to a zsig46 polypeptide, peptide or epitope with a binding affinity (Kₐ) of 10⁶ mol⁻¹ or greater, preferably 10⁷ mol⁻¹ or greater, more preferably 10⁸ mol⁻¹ or greater, and most preferably 10⁹ mol⁻¹ or greater. The binding affinity of an antibody can be readily determined by one of ordinary skill in the art, for example, by Scatchard analysis (Scatchard, G., Ann. NY Acad. Sci. 51: 660-672, 1949).

Second, antibodies specifically bind if they do not significantly cross-react with related polypeptides. Antibodies do not significantly cross-react with related polypeptide molecules, for example, if they detect a zsig46 polypeptide but not known related polypeptides using a standard Western blot analysis (Ausubel et al., ibid.). Examples of known related polypeptides are orthologs, proteins from the same species that are members of a protein family and the like. Moreover, antibodies may be “screened against” known related polypeptides to isolate a population that specifically binds to the inventive polypeptides. For example, antibodies raised to human zsig46 polypeptide are adsorbed to related polypeptides adhered to insoluble matrix; antibodies specific to human zsig46 polypeptide will flow through the matrix under the proper buffer conditions. Such screening allows isolation of polyclonal and monoclonal antibodies non-crossreactive to closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art (see, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984).

Genes encoding polypeptides having potential zsig46 polypeptide binding domains, “binding proteins”, can be obtained by screening random or directed peptide libraries displayed on phage (phage display) or on bacteria, such as E. coli. Nucleotide sequences encoding the polypeptides can be obtained in a number of ways, such as through random mutagenesis and random polynucleotide synthesis. Alternatively, constrained phage display libraries can also be produced. These peptide display libraries can be used to screen for peptides which interact with a known target which can be a protein or polypeptide, such as a ligand or receptor, a biological or synthetic macromolecule, or organic or inorganic substances. Techniques for creating and screening such peptide display libraries are known in the art (Ladner et al., U.S. Pat. Nos. 5,223,409; Ladner et al., 4,946,778; Ladner et al., 5,403,484 and Ladner et al., 5,571,698) and peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Peptide display libraries can be screened using the zsig46 sequences disclosed herein to identify proteins which bind to zsig46. These “binding proteins” which interact with zsig46 polypeptides can be used essentially like an antibody, for tagging cells; for isolating homolog polypeptides by affinity purification; directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. To increase the half-life of these binding proteins, they can be conjugated. Their biological properties may be modified by dimerizing or multimerizing for use as agonists or antagonists. Binding peptides can be screened against known related polypeptides as described above.

Antibodies and binding proteins to zsig46 may be used for tagging cells that express zsig46; for isolating zsig46 by affinity purification; for diagnostic assays for determining circulating levels of zsig46 polypeptides; for detecting or quantitating soluble zsig46 as marker of underlying pathology or disease; in analytical methods employing FACS; for screening expression libraries; for generating anti-idiotypic antibodies; and as neutralizing antibodies or as antagonists to block zsig46 polypeptide energy balance modulation activity or like activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Moreover, antibodies to zsig46 or fragments thereof may be used in vitro to detect denatured zsig46 or fragments thereof in assays, for example, Western Blots or other assays known in the art.

Antibodies or binding proteins herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zsig46 polypeptides or anti-zsig46 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anti-complementary pair, where the other member is bound to the polypeptide or antibody portion. For these purposes, biotin/streptavidin is an exemplary complementary/anti-complementary pair.

Northern blot analysis indicates that zsig46 is highly expressed in the thyroid gland. The thyroid gland is the major endocrine gland during normal human growth and development. In adults, the primary role of the thyroid is to maintain metabolic stability, primarily through thyroid hormone production and regulation. Virtually every organ in the body is affected by thyroid hormones. Thus, thyroid malfunction is associated with many disease states. Thyroid diseases are relatively common, occurring in the form of abnormalities in thyroid gland size and shape (goiter) and abnormalities in thyroid hormone secretion. Thyroid malfunction may also result from non-thyroidal illnesses or nutrient deficiencies that alter thyroid physiology. Examples of common thyroid diseases are thyrotoxicosis, hypothyroidism, Grave's disease, hyperthyroidism and thyroid tumors. For general review see, Felig, Baxter, and Frohman, (eds.), Endocrinology and Metabolism, McGraw Hill, NY, 3rd ed., 1995, pp. 432-553; and Bennett and Plum, (eds.), Textbook of Medicine, W. B. Saunders Co., Philadelphia, 20th ed., 1996, pp. 1227-45.

Thyroid hormones, such as thyroxine (T4), triiodothyronine (T3) and thyroid stimulating hormone (TSH), modulate a wide number of metabolic processes by regulating the production and activity of various enzymes, the production and metabolism of other hormones, and utilization of substrates, vitamins, and minerals. For review of the metabolic effect and diseases of thyroid hormones, see Braverman, (ed.), Diseases of the Thyroid, Humana Press, Totowa, N.J., 1997.

Thyroid expression of proteins of the present invention suggests a role in thyroid-related physiology and regulation. Such proteins act as regulatory proteins, like the thyroid hormones, and influence thyroid function or are secreted from thyroid with extrathymic effects. Thyroid proteins would be useful for, inter alia, restoring normal thyroid function in patients suffering various thyroid ailments and as targets for the development of small-molecule drugs. The molecules of the present invention may be useful for elucidation and prevention of various thyroid diseases and thyroid cancers. The polypeptides, nucleic acid and/or antibodies of the present invention may be used in treatment of disorders associated with thyroid dysfunction. The molecules of the present invention may used to modulate thyroid activity or to treat or prevent development of pathological conditions in thyroid tissue. In particular, certain syndromes and diseases may be amenable to such diagnosis, treatment or prevention.

The activity of molecules of the present invention may be measured using a variety of assays that measure thyroid function, including signal transduction upon binding a receptor, thyroid hormone secretion in vitro and in vivo or antibody binding. For example, zsig46 polypeptides can be labeled and tested for specific and saturating binding to specific cell lines or cells. After identification of positive cells to which zsig46 binds, activity can be tested for zsig46-mediated activation of a signal transduction pathway using methods known in the art. For instance, vector constructs containing a reporter (e.g. SRE-luciferase, STAT-luciferase, thyroid hormone response element (THRE)-luciferase or the like) can be introduced into the positive cell lines; such cell lines, when exposed to conditioned media containing secreted zsig46 protein, will demonstrate zsig46-mediated signal transduction activity through activation of the measurable reporter. Such assays are well known in the art. Specific assays include, but are not limited to, bioassays measuring signal transduction.

The molecules of the present invention may exert their effects in thyroid or extrathyroidally. Thus, activity of zsig46 polypeptides may affect thyroid function and can be measured by assessing thyroid hormone secretion in vitro or thyroid function in vivo. Thyroid ,hormones, and changes in thyroid hormone secretion in the presence or absence of zsig46, may be detected using various methods known in the art. For example, TSH and other thyroid hormones are commonly measured by radioimmune assay (RIA), immunometric assays (IMA) employing a “sandwich” type assay, or ELISA. Hormones, including T3 and T4, may be measured in vivo by a variety of methods known in the art (Elkins, Endocr. Rev., 11:5-46, 1990). For example, T4 may be measured using a radiometric ¹²⁵I binding assay, dialysis and ultrafiltration. See Braverman, L. E. (ed.), Diseases of the Thyroid, Humana Press, Totowa, N.J,, 1997, p.35-48.

Moreover, in vivo affects of zsig46 polypeptides on thyroid function can also be assessed in experimental animals or humans by ultrasound, radioactive iodine uptake, and fine needle aspiration biopsy (Braverman, (Ed.), ibid., p.35-48).

Thyroid malfunction and some of the currently associated therapies can elicit detrimental effects on bone in vivo. The effect of molecules of the present invention on bone may be determined using a variety of assays that measure bone function. Such assays are well known in the art.

Compounds identified as zsig46 agonists may be useful for promoting growth, proliferation or differentiation of various cell types in vitro and treatment of extrathyroid or thyroid disorders in vivo. For example, agonist compounds are useful as components of defined cell culture media, and may be used alone or in combination with other cytokines and hormones to replace serum that is commonly used in cell culture.

For pharmaceutical use, the proteins of the present invention are formulated for parenteral, particularly intravenous or subcutaneous, delivery according to conventional methods. Intravenous administration will be by bolus injection or infusion over a typical period of one to several hours. In general, pharmaceutical formulations will include a zsig46 protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton Pa., 19^{th} ed., 1995. Therapeutic doses will generally be determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years.

The invention is further illustrated by the following non-limiting examples.

### EXAMPLE 1

### Extension of EST Sequence

The novel zsig46 polypeptides of the present invention were initially identified by querying an EST database secretory signal sequences, characterized by an upstream methionine start site, a hydrophobic region of approximately 13 amino acids and a cleavage site, in an effort to select for secreted proteins. Polypeptides corresponding to ESTs meeting those search criteria were compared to known sequences to identify secreted proteins having homology to known ligands. A single EST sequence was discovered and predicted to be a secreted protein; however, no homology to known proteins was identified. A search of databases accepting data from the human genome project revealed that the EST polynucleotide sequence was present on human chromosome 13. Further analysis of the genomic DNA revealed a polynucleotide encoding a full length secreted zsig46 polypeptide, wherein the polynucleotide was characterized by two introns. A deletion was observed in the EST sequence, and a “C” residue was inserted at position 489 of SEQ ID NO:1 to replace the EST-deletion. The full length zsig46 polypeptide is encoded by DNA on human chromosome 13 at positions 23544 to 27547. The introns were located at base positions 23681-26472 and 26636-27310 of human chromosome 13 as submitted by Hawkins et al. The polynucleotide encoding zsig46, absent introns and including the position 489 insertion, is shown in SEQ ID NO:1.

The sequence of zsig46 was confirmed using the following procedure.

To identify the corresponding cDNA, a clone considered likely to contain the entire coding sequence of zsig46 polypeptide was used for sequencing. Using an Invitrogen S.N.A.P.™ Miniprep kit (Invitrogen, Corp., San Diego, Calif.) according to manufacturer's instructions a 5 ml overnight culture in LB+50 μg/ml ampicillin was prepared. The template was sequenced on an ABIPRISM™ model 377 DNA sequencer (Perkin-Elmer Cetus, Norwalk, Conn.) using the ABI PRISM™ Dye Terminator Cycle Sequencing Ready Reaction Kit (Perkin-Elmer Corp.) according to manufacturer's instructions. Oligonucleotides ZC976 (SEQ ID NO:3), ZC447 (SEQ ID NO:4) specific for the lacZ/clone-containing vector were used as sequencing primers. Oligonucleotides ZC14487 (SEQ ID NO: 5), ZC14716 (SEQ ID NO:6), ZC14712 (SEQ ID NO: 7), ZC14710 (SEQ ID NO: 8), ZC14488 (SEQ ID NO: 9) and ZC14711 (SEQ ID NO: 10) were used to complete the sequence from the clone. Sequencing reactions were carried out in a Hybaid OmniGene Temperature Cycling System (National Labnet Co., Woodbridge, N.Y.). SEQUENCHER™ 3.1 sequence analysis software (Gene Codes Corporation, Ann Arbor, Mich.) was used for data analysis. The resulting 1486 bp sequence is disclosed in SEQ ID NO. 1. Comparison of the originally derived EST sequence with the sequence represented in SEQ ID NO. 1 showed that there were 0 insertions, 0 deletions and 1 ambiguity resolved which resulted in 0 amino acid changes and 0 frame shifts between the deduced amino acid sequences.

### EXAMPLE 2

### Tissue Distribution

Northerns were performed using Human Multiple Tissue Blots from Clontech (Palo Alto, Calif.). A 420 base DNA probe was generated using primers within the coding region of the zsig46 gene shown in SEQ ID NO:1. The fragment was gel purified using a PCR purification kit (Qiagen, Inc., Chatsworth, Calif.) and was then radioactively labeled with ³²P random priming MEGAPRIME® DNA labeling system (Amersham, Arlington Heights, Ill.) according to the manufacturer's specifications. The probe was purified using a NUCTRAP push column (Stratagene Cloning Systems, La Jolla, Calif.). EXPRESSHYB (Clontech, Palo Alto, Calif.) solution was used for prehybridization and as a hybridizing solution for the Northern blots. Hybridization took place overnight at 65° C. using 1×10⁶ cpm/ml of labeled probe. The blots were then washed in solution I (2×SSC and 0.05% SDS) at 25° C. for 45 minutes, followed by a wash in solution II (0.1×SSC and 0.1% SDS) at 50° C. for 1 hour. The blots were exposed to film for 4 hours. The resulting blots were characterized by high background. Consequently, the blots were washed again at 55° C. in solution II for 90 minutes and exposed to film overnight. Three transcript sizes were observed at approximately 1, 2 and 3 kb. Signal intensity indicated that zsig46 polypeptide was predominantly expressed in the thyroid. Weak expression was also observed in adrenal gland, trachea, lymph node, spinal cord, small intestine and ovary.

### EXAMPLE 3

### Chromosomal Mapping of the Zsiq46 Gene

The zsig46 gene was mapped to chromosome 13 using the commercially available “GeneBridge 4 Radiation Hybrid Panel” (Research Genetics, Inc., Huntsville, Ala.). The GeneBridge 4 Radiation Hybrid Panel contains PCRable DNAs from each of 93 radiation hybrid clones, plus two control DNAs (the HFL donor and the A23 recipient). A publicly available WWW server (http://www-genome.wi.mit. edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of the zsig46 gene with the “GeneBridge 4 RH Panel”, 20 μl reactions were set up in a PCRable 96-well microtiter plate (Stratagene, La Jolla, Calif.) and used in a “RoboCycler Gradient 96” thermal cycler (Stratagene). Each of the 95 PCR reactions consisted of 2 μl 10×KlenTaq PCR reaction buffer (Clontech), 1.6 μl dNTPs mix (2.5 mM each, Perkin-Elmer, Foster City, Calif.), 1 μl sense primer (SEQ ID NO: 11), 1 μl antisense primer (SEQ ID NO: 12), 2 μl RediLoad (Research Genetics, Inc.), 0.4 μl 50×Advantage KlenTaq Polymerase Mix (Clontech), 25 ng of DNA from an individual hybrid clone or control and ddH₂O for a total volume of 20 μl. The reactions were overlaid with an equal amount of mineral oil and sealed. The PCR cycler conditions were as follows: an initial 1 cycle 5 minute denaturation at 95° C., 35 cycles of a 1 minute denaturation at 95° C., 1 minute annealing at 60° C. and 1.5 minute extension at 72° C., followed by a final 1 cycle extension of 7 minutes at 72° C. The reactions were separated by electrophoresis on a 3% NuSieve GTG agarose gel (FMC Bioproducts, Rockland, Me.).

The results showed that the zsig46 gene maps 217.1 cR_{—}3000 from the top of the human chromosome 13 linkage group on the WICGR radiation hybrid map. Proximal and distal framework markers were AFM350XA5 and CHLC.GATA30F02 (D13S789), respectively. The use of surrounding markers positions Zsig46 in the 13q22.2-q22.3 region on the integrated LDB chromosome 13 map (The Genetic Location Database, University of Southhampton, WWW server: http://cedar.genetics.soton.ac.uk/public_html/).

### EXAMPLE 4

### Mammalian Expression Vectors zsiq46CEE/pZP9 and zsiq46NEE/pZP9

Two expression vectors were prepared for the zsig46 polypeptide, zsig46CEE/pZP9 and zsig46NEE/pZP9, wherein the constructs are designed to express a zsig46 polypeptide with a C- or N-terminal Glu-Glu tag (SEQ ID NO:14).

Zsig46CEE/pZP9

A PCR generated zsig46 DNA fragment was created using ZC14695 (SEQ ID NO:15) and ZC15231 (SEQ ID NO:16) as PCR primers and the template described in Example 1 above which incorporated a 5′ Xho I site and a 3′ Bam HI site and Glu-Glu tag. The resultant PCR product was digested with the restriction enzymes Bam HI and Xho I and visualized by gel electrophoresis. A band of the predicted size, 1025 bp, was excised and the DNA was purified from the gel with a QUIAQUICK® column (Qiagen) according the manufacturer's instructions.

The excised DNA was subcloned into plasmid pZP9 which had been cut with Bam HI and Xho I. The zsig46CEE/pZP9 expression vector contains a sequence encoding a Glu-Glu tag (SEQ ID NO:14) which is attached at the C-terminus of the inserted sequence as a purification aid, and also uses the native zsig46 signal sequence. Plasmid pZP9 (deposited at the American Type Culture Collection, 12301 Parklawn Drive, Rockville, Md., ATCC #98668) is a mammalian expression vector containing an expression cassette having the mouse metallothionein-1 promoter, multiple restriction sites for insertion of coding sequences, a stop codon and a human growth hormone terminator. The plasmid also has an E. coli origin of replication, a mammalian selectable marker expression unit having an SV40 promoter, enhancer and origin of replication, a DHFR gene and the SV40 terminator.

zsig46NEE/pZP9

A PCR generated zsig46 DNA fragment was created using ZC14696 (SEQ ID NO:17) and ZC15230 (SEQ ID NO:18) as PCR primers and the template described in Example 1 above which incorporated a 5′ Bam HI site and Glu-Glu tag and a 3′ Xho I site. The resultant PCR product was digested with the restriction enzymes plasmid NEE/pZP9 which had been cut with Bam HI and Xho I. The resultant PCR product was digested with the restriction enzymes Bam HI and Xho I and visualized by gel electrophoresis. A band of the predicted size, 1099 bp, was excised and the DNA was purified from the gel with a QUIAQUICK® column (Qiagen) according the manufacturer's instructions.

The excised DNA was subcloned into plasmid pZP9 which had been cut with Bam HI and Xho I. The zsig46/NEEpZP9 expression vector and attaches the Glu-Glu tag (SEQ ID NO:14) to the N-terminal of the zsig46 polypeptide-encoding polynucleotide sequence. Plasmid pZP9 (deposited at the American Type Culture Collection, 12301 Parklawn Drive, Rockville, Md.) is a mammalian expression vector containing an expression cassette having the mouse metallothionein-1 promoter, a TPA leader peptide, multiple restriction sites for insertion of coding sequences, and a human growth hormone terminator. The plasmid also contains an E. coli origin of replication, a mammalian selectable marker expression unit having an SV40 promoter, enhancer and origin of replication, a DHFR gene and the SV40 terminator.

A 1:4 ratio of the restriction digested zsig46 insert to corresponding vector was ligated at room temperature for 4 hours. One microliter of each ligation reaction was independently electroporated into DH10B competent cells (GIBCO BRL, Gaithersburg, Md.) according to manufacturer's direction and plated onto LB plates containing 50 mg/ml ampicillin, and incubated overnight. Sequences of positive clones were verified by sequence analysis. A large scale plasmid preparation was done using a QIAGEN® Maxi prep kit (Qiagen) according to manufacturer's instructions.

### EXAMPLE 5

### Construction of Baculovirus Expression Vectors

Two expression vectors were prepared to express zsig46 polypeptides in insect cells. A 1087 bp zsig46CEE fragment was excised from zsig46CEE/pHZ9 and a 1135 bp zsig46NEE fragment was excised from zsig46NEE/pHZ9 using restriction enzymes Eco RI and Xba I and visualized by gel electrophoresis. The bands were excised, purified, and ligated into an Eco RI/Xba I digested baculovirus expression vector derived from pFastBac1™ (Life Technologies). A 1:4 ratio of the restriction digested zsig46CEE and zsig46NEE inserts to vector were ligated overnight. Four fmol of the diluted ligation mix was transformed into DH5α Library Efficiency competent cells (Life Technologies) according to manufacturer's direction by heat shock for 45 seconds in a 42° C. waterbath. The ligated DNA was diluted in 450 μl SOC media (2% Bacto Tryptone, 0.5% Bacto Yeast Extract, 10 ml 1M NaCl, 1.5 mM KCl, 10 mM MgCl₂, 10 mM MgSO₄ and 20 mM glucose) and plated onto LB plates containing 100 μg/ml ampicillin. The plates were incubated overnight at 37° C. Plasmid DNA was prepared using the QiaVac Miniprep8 system (Qiagen) according the manufacturer's directions. Clones containing the correct insert were subject to large scale plasmid preparation was done using a QIAGEN® Maxi prep kit (Qiagen) according to manufacturer's instructions.

One microliter of each of the above constructs was used to independently transform 10 μl DH10Bac Max Efficiency competent cells (GIBCO-BRL, Gaithersburg, Md.) according to manufacturer's instruction, by heat shock at 42° C. for 45 seconds. The transformants were then diluted in 980 μl SOC media and plated on to Luria Agar plates containing 50 μg/ml kanamycin, 7 μg/ml gentamicin, 10 μg/ml tetracycline, IPTG and Bluo Gal. The cells were incubated for 48 hours at 37° C. A color selection was used to identify those cells having virus that had incorporated into the plasmid (referred to as a “bacmid”). Those colonies, which were white in color, were picked for analysis. Bacmid DNA was isolated from positive. Those having the correct insert were used to transfect Spodoptera frugiperda (Sf9) cells.

Sf9 cells were seeded at 5×10⁶ cells per 35 mm plate and allowed to attach for 1 hour at 27° C. Five microliters of bacmid DNA was diluted with 100 μl Sf-900 II SFM. Six microliters of CellFECTIN Reagent (Life Technologies) was diluted with 100 μl Sf-900 II SMF. The bacmid DNA and lipid solutions were gently mixed and incubated 30-45 minutes at room temperature. The media from one plate of cells were aspirated, and the lipid-DNA mixture to which 0.8 ml of Sf-900 II SFM was added. The cells were incubated at 27° C. for 4 hours, then 2 ml of Sf-900 II media containing penicillin/streptomycin was added to each plate. The plates were incubated at 27° C., 90% humidity, for 96 hours after which the virus was harvested.

Primary Amplification

Sf9 cells were grown in 50 ml Sf-900 II SFM in a shake flask to an approximate density of 0.50×10⁶ cells/ml. The cells were then transfected with 200 μl of the virus stock from above and incubated at 27° C. for 3 days after which time the virus was harvested, titer ranged from 4.6 to 6.5×10⁷ pfu/ml. To scale up, SF9 cells were grown for 74 hours to a density of 1.2×10⁶ cells/ml in five liters of SF 900 II SFM. The cells were then transfected with the harvested virus and incubated as above for 47 hours.

### EXAMPLE 6

### Purification Conditions for zsiq46 NEE and CEE From Baculovirus Infected Sf9 Cells

Unless otherwise noted, all operations were carried out at 4° C. A mixture of protease inhibitors was added to a 2000 ml sample of conditioned media from baculovirus-infected Sf9 cells to final concentrations of 2.5 mM ethylenediaminetetraacetic acid (EDTA, Sigma Chemical Co. St. Louis, Mo.), 0.001 mM leupeptin (Boehringer-Mannheim, Indianapolis, Ind.), 0.001 mM pepstatin (Boehringer-Mannheim) and 0.4 mM Pefabloc (Boehringer-Mannheim). The sample was centrifuged at 10,000 rpm for 30 min at 4° C. in a Beckman JLA-10.5 rotor (Beckman Instruments, Palo Alto, Calif.) in a Beckman Avanti J25I centrifuge (Beckman Instruments) to remove cell debris. To the supernatant fraction was added a 50.0 ml sample of anti-EE Sepharose (prepared as described below), and the mixture was gently agitated on a Wheaton (Millville, N.J.) roller culture apparatus for 18.0 h at 4° C.

The mixture was poured into a 5.0×20.0 cm Econo-Column (Bio-Rad, Laboratories, Hercules, Calif.) and the gel was washed with 30 column volumes of phosphate buffered saline (PBS). The unretained flow-through fraction was discarded. Once the absorbance of the effluent at 280 nM was less than 0.05, flow through the column was reduced to zero and the anti-EE Sepharose gel was washed with 2.0 column volumes of PBS containing 0.2 mg/ml of EE peptide (AnaSpec, San Jose, Calif.). The peptide used has the sequence N-Glu-Tyr-Met-Pro-Val-Asp (SEQ ID NO:15). After 1.0 h at 4° C., flow was resumed and the eluted protein was collected. This fraction is referred to as the peptide elution. The anti-EE Sepharose gel was washed with 2.0 column volumes of 0.1M glycine, pH 2.5, and the glycine wash was collected separately. The pH of the glycine-eluted fraction was adjusted to 7.0 by the addition of a small volume of 10×PBS and stored at 4° C. for future analysis if needed.

The peptide elution was concentrated to 5.0 ml using a 5,000 molecular weight cutoff membrane concentrator (Millipore, Bedford, Mass.) according to the manufacturer's instructions. The concentrated peptide elution was separated from free peptide by chromatography on a 1.5×50 cm Sephadex G-50 (Pharmacia, Piscataway, N.J.) column equilibrated in PBS at a flow rate of 1.0 ml/min using a BioCad Sprint HPLC (PerSeptive BioSystems, Framingham, Mass.). Two-ml fractions were collected and the absorbance at 280 nM was monitored. The first peak of material absorbing at 280 nM and eluting near the void volume of the column was collected. This material represented purified zsig46 NEE or zsig46 CEE and was further characterized by SDS-PAGE and Western blotting with anti-EE antibodies.

On Coomassie Blue stained SDS-PAGE gels under non-reducing conditions, the zsig46 NEE was composed of a closely spaced doublet of apparent molecular weights 53,000 and 51,000. Only the larger molecular weight showed cross-reactivity with anti-EE antibodies. Under reducing conditions, the mobility of these bands was slightly decreased and the reduced material migrated with apparent molecular weight of 55,000 and 53,000. Again, only the larger molecular weight band showed cross-reactivity on Western blots with the anti-EE antibody.

For zsig46CEE, electrophoresis on Coomassie Blue-stained SDS-PAGE gels showed one major protein band of apparent molecular weight 52,000. Reducing agents had no affect on the mobility of this band. By Western blot analysis, the 52,000 Da band showed cross-reactivity with anti-EE antibodies. The protein concentration of the purified proteins was performed by BCA analysis (Pierce, Rockford, Ill.) and the material was aliquoted, and stored at −80° C. according to our standard procedures. The concentrations of zsig46 NEE and zsig46 CEE was 0.27 mg/ml and 1.0 mg/ml, respectively.

Preparation of anti-EE Sepharose

A 100 ml bed volume of protein G-Sepharose (Pharmacia, Piscataway, N.J.) was washed 3 times with 100 ml of PBS containing 0.02% sodium azide using a 500 ml Nalgene 0.45 micron filter unit. The gel was washed with 6.0 volumes of 200 mM triethanolamine, pH 8.2 (TEA, Sigma, St. Louis, Mo.) and an equal volume of EE antibody solution containing 900 mg of antibody was added. After an overnight incubation at 4° C., unbound antibody was removed by washing the resin with 5 volumes of 200 mM TEA as described above. The resin was resuspended in 2 volumes of TEA, transferred to a suitable container, and dimethylpimilimidate-2HCl (Pierce, Rockford, Ill.), dissolved in TEA, was added to a final concentration of 36 mg/ml of gel. The gel was rocked at room temperature for 45 min and the liquid was removed using the filter unit as described above. Nonspecific sites on the gel were then blocked by incubating for 10 min at room temperature with 5 volumes of 20 mM ethanolamine in 200 mM TEA. The gel was then washed with 5 volumes of PBS containing 0.02% sodium azide and stored in this solution at 4° C.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2.

2. An isolated polypeptide according to claim 1, wherein said polypeptide is at least 90% identical in amino acid sequence to residues 29-346 of SEQ ID NO:2, wherein said sequence comprises cysteine residues corresponding to residues 58, 65, 132, 147, 153 and 219 of SEQ ID NO:2.

3. An isolated polypeptide according to claim 2, wherein said polypeptide comprises residues 1-346 of SEQ ID NO:2.

4. An isolated polypeptide according to claim 1, covalently linked amino terminally or carboxy terminally to a moiety selected from the group consisting of: affinity tags, toxins, radionucleotides, enzymes and fluorophores.

5. An isolated polypeptide having a sequence of amino acid residues selected from the group consisting of:
a) a sequence of amino acid residues from amino acid residue 1 to amino acid residue 37 of SEQ ID NO: 2;
b) a sequence of amino acid residues from amino acid residue 29 to amino acid residue 37 of SEQ ID NO: 2;
c) a sequence of amino acid residues from amino acid residue 31 to amino acid residue 37 of SEQ ID NO: 2;
d) a sequence of amino acid residues from amino acid residue 29 to amino acid residue 45 of SEQ ID NO: 2;
e) a sequence of amino acid residues from amino acid residue 31 to amino acid residue 45 of SEQ ID NO: 2;
f) a sequence of amino acid residues from amino acid residue 40 to amino acid residue 45 of SEQ ID NO: 2;
g) a sequence of amino acid residues from amino acid residue 40 to amino acid residue 346 of SEQ ID NO: 2;
h) a sequence of amino acid residues from amino acid residue 48 to amino acid residue 346 of SEQ ID NO: 2;
i) a sequence of amino acid residues from amino acid residues 29 to amino acid residue 276 of SEQ ID NO:2;
j) a sequence of amino acid residues from amino acid residues 31 to amino acid residue 276 of SEQ ID NO:2;
k) a sequence of amino acid residues from amino acid residues 40 to amino acid residue 276 of SEQ ID NO:2;
l) a sequence of amino acid residues from amino acid residues 48 to amino acid residue 276 of SEQ ID NO:2;
m) a sequence of amino acid residues from amino acid residue 278 to amino acid residue 346 of SEQ ID NO: 2; and
n) a sequence of amino acid residues that is at least 80% identical in amino acid sequence to a), b), c), d), e), f), g), h), i), j), k), l) or m).

6. A fusion protein consisting essentially of a first portion and a second portion joined by a peptide bond, said first portion comprising a polypeptide that is at least 80% identical in amino acid sequence to the amino acid sequence of a polypeptide selected from the group consisting of:
a) a sequence of amino acid residues from amino acid residue 1 to amino acid residue 37 of SEQ ID NO: 2;
b) a sequence of amino acid residues from amino acid residue 29 to amino acid residue 37 of SEQ ID NO: 2;
c) a sequence of amino acid residues from amino acid residue 31 to amino acid residue 37 of SEQ ID NO: 2;
d) a sequence of amino acid residues from amino acid residue 29 to amino acid residue 45 of SEQ ID NO: 2;
e) a sequence of amino acid residues from amino acid residue 31 to amino acid residue 45 of SEQ ID NO: 2;
f) a sequence of amino acid residues from amino acid residue 40 to amino acid residue 45 of SEQ ID NO: 2;
g) a sequence of amino acid residues from amino acid residue 40 to amino acid residue 346 of SEQ ID NO: 2;
h) a sequence of amino acid residues from amino acid residue 48 to amino acid residue 346 of SEQ ID NO: 2;
i) a sequence of amino acid residues from amino acid residues 29 to amino acid residue 276 of SEQ ID NO:2;
j) a sequence of amino acid residues from amino acid residues 31 to amino acid residue 276 of SEQ ID NO:2;
k) a sequence of amino acid residues from amino acid residues 40 to amino acid residue 276 of SEQ ID NO:2;
l) a sequence of amino acid residues from amino acid residues 48 to amino acid residue 276 of SEQ ID NO:2;
m) a sequence of amino acid residues from amino acid residue 278 to amino acid residue 346 of SEQ ID NO: 2;
o) a sequence of amino acid residues from amino acid residue 1 to amino acid residue 346 of SEQ ID NO: 2;
p) a sequence of amino acid residues from amino acid residue 29 to amino acid residue 346 of SEQ ID NO: 2; and
q) a sequence of amino acid residues from amino acid residue 31 to amino acid residue 346 of SEQ ID NO: 2; and
said second portion comprising another polypeptide.

7. A fusion protein comprising a secretory signal sequence having the amino acid sequence of amino acid residues 1-28 or 1-30 of SEQ ID NO:2, wherein said secretory signal sequence is operably linked to an additional polypeptide.

8. An isolated protein comprising a first polypeptide that is at least 80% identical in amino acid sequence to the amino acid sequence of a polypeptide selected from the group consisting of:
a) amino acid residues 1-29 of SEQ ID NO:2, said polypeptide comprising a cysteine residue at a position corresponding to residue 15 of SEQ ID NO:2;
b) amino acid residues 1-30 of SEQ ID NO:2, said polypeptide comprising a cysteine residue at a position corresponding to residue 15 of SEQ ID NO:2;
c) amino acid residues 48-276 of SEQ ID NO:2, said polypeptide comprising cysteine residues at positions corresponding to residues 58, 65, 132, 147, 153 and 219 of SEQ ID NO:2; and
d) amino acid residues 31-346 of SEQ ID NO:2, said polypeptide comprising cysteine residues at positions corresponding to residues 58, 65, 132, 147, 153 and 219 of SEQ ID NO:2;
complexed to a second polypeptide.

9. An isolated protein according to claim 8, wherein said first polypeptides and said second polypeptide are complexed by intermolecular disulfide bonds.

10. An isolated protein according to claim 9, wherein said protein is a homodimer.

11. An isolated protein according to claim 9, wherein said protein is a heterodimer.

12. An expression vector comprising the following operably linked elements:
a transcription promoter;
a DNA segment encoding a polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2; and
a transcription terminator.

13. An expression vector according to claim 12, wherein said DNA segment encodes a polypeptide that is at least 90% identical in amino acid sequence to residues 29-346 of SEQ ID NO:2 wherein said sequence comprises cysteine residues corresponding to residues 58, 65, 132, 147, 153 and 219 of SEQ ID NO:2.

14. An expression vector according to claim 13, wherein said DNA segment encodes a polypeptide comprising residues 1-346 of SEQ ID NQ:2.

15. An expression vector according to claim 12, wherein said DNA segment encodes a polypeptide covalently linked amino terminally or carboxy terminally to an affinity tag.

16. An expression vector according to claim 12 wherein said DNA segment further encodes a secretory signal sequence operably linked to said polypeptide.

17. An expression vector according the claim 16, wherein said secretory signal sequence comprises residues 1-28 or 1-30 of SEQ ID NO:2.

18. A cultured cell into which has been introduced an expression vector comprising the following operably linked elements:
a transcription promoter;
a DNA segment encoding a polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2; and
a transcription terminator, wherein said cell expresses said polypeptide encoded by said DNA segment.

19. A method of producing a polypeptide comprising:
culturing a cell into which has been introduced an expression vector comprising the following operably linked elements:
a transcription promoter;
a DNA segment encoding a polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2; and
a transcription terminator;
whereby said cell expresses said polypeptide encoded by said DNA segment; and
recovering said expressed polypeptide.

20. A pharmaceutical composition comprising a polypeptide, said polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2;
in combination with a pharmaceutically acceptable vehicle.

21. An antibody that specifically binds to an epitope of a polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2.

22. A binding protein that specifically binds to an epitope of a polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2.

23. An isolated polynucleotide encoding a polypeptide comprising a sequence of amino acid residues that is at least 80% identical in amino acid sequence to residues 31-346 of SEQ ID NO:2, wherein said sequence comprises cysteine residues corresponding to residues 58, 65, 132, 147, 153 and 219 of SEQ ID NO:2.

24. An isolated polynucleotide according to claim 23, wherein said polypeptide is at least 90% identical in amino acid sequence to residues 29-346 of SEQ ID NO:2 wherein said sequence comprises cysteine residues corresponding to residues 58, 65, 132, 147, 153 and 219 of SEQ ID NO:2.

25. An isolated polynucleotide according to claim 24, wherein said polypeptide comprises residues 1-346 of SEQ ID NO:2.

26. An isolated polynucleotide according to claim 23, wherein said polynucleotide is DNA.

27. An isolated polynucleotide selected from the group consisting of:
a) a sequence of nucleotides from nucleotide 47 to nucleotide 157 of SEQ ID NO:1;
b) a sequence of nucleotides from nucleotide 131 to nucleotide 157 of SEQ ID NO:1;
c) a sequence of nucleotides from nucleotide 137 to nucleotide 157 of SEQ ID NO:1;
d) a sequence of nucleotides from nucleotide 131 to nucleotide 181 of SEQ ID NO:1;
e) a sequence of nucleotides from nucleotide 137 to nucleotide 181 of SEQ ID NO:1;
f) a sequence of nucleotides from nucleotide 164 to nucleotide 1084 of SEQ ID NO:1;
g) a sequence of nucleotides from nucleotide 188 to nucleotide 1084 of SEQ ID NO:1;
h) a sequence of nucleotides from nucleotide 164 to nucleotide 181 of SEQ ID NO:1;
i) nucleotide sequences complementary to a), b), c), d), e), f), g) or h); and
j) degenerate nucleotide sequences of a), b), c), d), e), f), g), h) or i).

28. An isolated polynucleotide selected from the group consisting of:
a) a sequence of nucleotides from nucleotide 131 to nucleotide 874 of SEQ ID NO:1;
b) a sequence of nucleotides from nucleotide 137 to nucleotide 874 of SEQ ID NO:1;
c) a sequence of nucleotides from nucleotide 164 to nucleotide 874 of SEQ ID NO:1;
d) a sequence of nucleotides from nucleotide 188 to nucleotide 874 of SEQ ID NO:1;
e) a sequence of nucleotides from nucleotide 878 to nucleotide 1084 of SEQ ID NO:1;
f) nucleotide sequences complementary to a), b), c), d) or e) and
g) degenerate nucleotide sequences of a), b), c), d), e) or f).

29. An isolated polynucleotide encoding a fusion protein consisting essentially of a first portion and a second portion joined by a peptide bond, said first portion is a polypeptide that is at least 80% identical in amino acid sequence to the amino acid sequence of a polypeptide selected from the group consisting of:
a) a sequence of amino acid residues from amino acid residue 1 to amino acid residue 37 of SEQ ID NO: 2;
b) a sequence of amino acid residues from amino acid residue 29 to amino acid residue 37 of SEQ ID NO: 2;
c) a sequence of amino acid residues from amino acid residue 31 to amino acid residue 37 of SEQ ID NO: 2;
d) a sequence of amino acid residues from amino acid residue 29 to amino acid residue 45 of SEQ ID NO: 2;
e) a sequence of amino acid residues from amino acid residue 31 to amino acid residue 45 of SEQ ID NO: 2;
f) a sequence of amino acid residues from amino acid residue 40 to amino acid residue 45 of SEQ ID NO: 2;
g) a sequence of amino acid residues from amino acid residue 40 to amino acid residue 346 of SEQ ID NO: 2;
h) a sequence of amino acid residues from amino acid residue 48 to amino acid residue 346 of SEQ ID NO: 2;
i) a sequence of amino acid residues from amino acid residues 29 to amino acid residue 276 of SEQ ID NO:2;
j) a sequence of amino acid residues from amino acid residues 31 to amino acid residue 276 of SEQ ID NO:2;
k) a sequence of amino acid residues from amino acid residues 40 to amino acid residue 276 of SEQ ID NO:2;
l) a sequence of amino acid residues from amino acid residues 48 to amino acid residue 276 of SEQ ID NO:2;
m) a sequence of amino acid residues from amino acid residue 278 to amino acid residue 346 of SEQ ID NO: 2;
o) a sequence of amino acid residues from amino acid residue 1 to amino acid residue 346 of SEQ ID NO: 2;
p) a sequence of amino acid residues from amino acid residue 29 to amino acid residue 346 of SEQ ID NO: 2; and
q) a sequence of amino acid residues from amino acid residue 31 to amino acid residue 346 of SEQ ID NO: 2; and
said second portion comprising another polypeptide.

30. An isolated polynucleotide encoding a fusion protein comprising a secretory signal sequence having the amino acid sequence of amino acid residues 1-28 or 1-30 of SEQ ID NO:2, wherein said secretory signal sequence is operably linked to an additional polypeptide.

31. An isolated polynucleotide comprising the sequence of nucleotide 1 to nucleotide 1084 of SEQ ID NO:13.

32. An oligonucleotide probe or primer comprising at least 14 contiguous nucleotides of a polynucleotide of SEQ ID NO:13 or a sequence complementary to SEQ ID NO:13.

33. A method for detecting a genetic abnormality in a patient, comprising:
obtaining a genetic sample from a patient;
incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence, to produce a first reaction product;
comparing said first reaction product to a control reaction product, wherein a difference between said first reaction product and said control reaction product is indicative of a genetic abnormality in the patient.
